(19) United States
(12) Patent Application Publication
(10) Pub. No.: US 2026/0286309 A1
(43) Pub. Date: Sep. 24, 2026

(54) DIFFERENTIATION OF IPSCS IN BIOREACTORS

(71) Applicant: REPAIRON GMBH, Göttingen (DE)

(72) Inventor: Luis HAUPT, Göttingen (DE)

(73) Assignee: REPAIRON GMBH, Göttingen (DE)

(21) Appl. No.: 18/881,214

(22) PCT Filed: Jul. 5, 2023
(86) PCT No.: PCT/EP2023/068492

§ 371 Date: Jan. 3, 2025

(30) Foreign Application Priority Data
Jul. 5, 2022 (EP) 22183054.0

(51) Int. Cl. C12N 5/077

(52) U.S. Cl. CPC C12N 5/0657; C12N 2501/115; C12N 2501/155; C12N 2501/16; C12N 2501/727; C12N 2506/45

(57) ABSTRACT

The present invention relates to a method of producing a population of differentiated cells from pluripotent stem cells (PSC) in suspension culture in a bioreactor, the method comprising (i) cultivating the PSC under suitable conditions to allow mesodermal induction, (ii) inducing differentiation under suitable conditions of the PSC of step (i), and (iii) conducting metabolic selection under suitable conditions of the PSC of step (ii), wherein steps (i) to (iii) are conducted in a closed bioreactor system, thereby producing a population of differentiated cells. The present invention further relates to a population of differentiated cells obtainable or obtained by said method. The present invention further describes a method of dissociating cell aggregates in a closed bioreactor system.

### CROSS-REFERENCE TO RELATED APPLICATIONS

The present application claims the benefit of priority of European Patent Application No. 22183054.0 filed 5 Jul. 2022, the content of which is hereby incorporated by reference it its entirety for all purposes.

### TECHNICAL FIELD OF THE INVENTION

The present invention describes a process of producing of large amounts of induced pluripotent stem cell-derived cells in bioreactors. Accordingly, the present invention relates to a method of producing a population of differentiated cells from pluripotent stem cells (PSC) in suspension culture in a bioreactor, the method comprising (i) cultivating the PSC under suitable conditions to allow mesodermal induction, (ii) inducing differentiation under suitable conditions of the PSC of step (i), and (iii) optionally selecting the PSC of step (ii), wherein steps (i) to (iii) are conducted in a closed bioreactor system, thereby producing a population of differentiated cells. The present invention further describes a population of differentiated cells obtainable or obtained by said method. The present invention further describes a method of dissociating cell aggregates in a closed bioreactor system.

### BACKGROUND

In basic research, with less demand for large amounts of cells, induced pluripotent stem cells (iPSC) and iPSC-derived cells are routinely grown as adherent cell culture. Here, the cells attach to the surface of a culture dish and grow as colonies or a monolayer. The adherent cell culture of iPSCs and iPSC-derived cells is not suitable for the generation of large amounts of cells that are needed for clinical applications. This is because it is material- and labor-intensive. Furthermore, the outcome and quality of the cell production highly depends on the operator because the process is usually not automated and only not fully monitored and controlled.

It has been reported that the use of bioreactor systems enables production of large amounts of iPSCs and iPSC-derived cells (Kropp et al., 2017). In these systems, iPSCs and iPSC-derived cells usually do not attach to the surface of a dish but are grown in a free-floating suspension because iPSCs form aggregates when cultivated in suspension. Suspension culture in bioreactor systems is described to be more efficient than adherent culture because the culture can be monitored, controlled and automated even at high cell numbers and less material and amount of work is needed. Importantly, for these reasons the use of bioreactor systems would be preferred over static culture for good manufacturing practice (GMP)-controlled applications. Different bioreactor systems have been reported for suspension culture of iPSCs with stirred tank reactor (STR) systems being the best described ones. It was shown that high numbers of iPSCs can be successfully generated in stirred bioreactor (STR) systems (Chen et al., 2012, 2015; Halloin et al., 2019; Hemmi et al., 2014; Jiang et al., 2019; Kempf et al., 2015; Kropp et al., 2016; Le and Hasegawa, 2019).

Ideally, a large-scale production of PSC-derived cells for clinical applications under GMP can be performed in a closed system. However, there still remain technical issues that so far prevent the broad application of closed systems for the production of PSC-derived cells.

Accordingly, there still is a need for methods for producing a population of differentiated cells in large quantities. The technical problem therefore is to comply with this need.

### SUMMARY OF THE INVENTION

The invention describes a process for the large-scale production of iPSC-derived cardiomyocytes (iPSC-CM) in STRs in which the entire culture starting from inoculation of iPSCs and ending with the harvest of iPSC-CMs, including iPSC aggregate formation, iPSC expansion and cardiac differentiation, is performed in a closed system without the need of manual interference. Such a process is ideal for a large-scale production of iPSC-CMs under GMP for clinical application with a high level of automatization and control. The technical problem is solved by the subject-matter as defined in the claims.

Accordingly, the present invention relates to a method of producing a population of differentiated cells from pluripotent stem cells (PSC) in suspension culture in a bioreactor, the method comprising
(i) cultivating the PSC under suitable conditions to allow mesodermal induction,
(ii) inducing differentiation under suitable conditions of the PSC of step (i), and
(iii) optionally selecting the PSC of step (ii),
   wherein steps (i) to (iii) are conducted in a closed bioreactor system, thereby producing a population of differentiated cells.

The method of the invention may further comprise step (0) of expanding PSC under suitable conditions.

In the method of the invention, after step (ii) a further step (ii) (a) may be conducted of expanding the PSC of step (ii) under suitable conditions.

In the method of the invention, after step (iii) a further step (iv) may be conducted of recovery of the PSC of step (iii) under suitable conditions in the closed bioreactor system. In the method of the invention, after step (ii), iii) or (iv) a further step (v) is preferably conducted of harvesting of the population of differentiated cells.

The harvesting of the population of differentiated cells may include dissociating aggregates formed during any one of steps (i) to (iv).

Dissociating aggregates may include
(a) allowing the cells or aggregates to sediment at the bottom of the closed bioreactor system and removing the supernatant;
(b) adding a cell dissociation agent, preferably an enzyme such as trypsin;
(c) agitating the cells or cell aggregates;
(d) repeating steps (a)-(c) three times; and
(e) stopping the cell dissociation by adding stop medium, preferably wherein the stop medium comprises Knockout-Serum replacement,
   preferably wherein the differentiated cells are cardiomyocytes.

Enriching in step (iii) may comprise conducting metabolic selection under suitable conditions.

The closed bioreactor system preferably is a stirred bioreactor, a rocking motion bioreactor and/or a multi parallel bioreactor.

The media preferably are
in step (0) iPS-brew basal medium comprising iPS-brew supplement;
in step (i) a mesodermal induction medium (MIM), which comprises RPMI-1640, about 1 to 5% B27 minus insulin, about 100 to 300 μmol/L l-ascorbic acid-2-phosphate sesquimagnesium salt hydrate, about 0.1 to 10 mM sodium pyruvate, about 5 to 15 ng/mL Activin A, about 1 to 10 ng/ml BMP4, about 1 to 10 ng/ml bFGF, optionally about 1 to 3 μM CHIR99021;
in step (ii) a cardiomyocyte induction medium (CM-IM) comprising RPMI-1640, about 1 to 5% B27, about 100 to 300 μmol/L I-ascorbic acid-2-phosphate sesquimagnesium salt hydrate, about 0.1 to 10 mM sodium pyruvate, about 1 to 15 μM IWP4;
in step (iii) a selection medium comprising RPMI-1640 minus glucose, about 1 to 5 mM Lactate, about 0.01 to 0.5 mM 2-Mercaptoethanol, about 2 to 8 mM HEPES; and
in step (iv) a basal serum-free medium (BSFM), which comprises RPMI-1640, about 1 to 5% B27, about 100 to 300 μmol/L L-ascorbic acid-2-phosphate sesquimagnesium salt hydrate, and about 0.1 to 10 mM sodium pyruvate.

In the method of the invention, the steps preferably are conducted for the following periods
step (0) optionally about 4 to 6 days,
step (i) about 24 hours to 5 days,
step (ii) about 4 to 10 days,
step (ii) (a) about 1 to 5 days,
step (iii) about 5 to 10 days,
step (iv) about 12 hours to 5 days.

A change of the medium preferably is conducted at least at the transition from step (0) to step (i), from step (i) to step (ii), from step (ii) to step (ii) (a), from step (ii) (a) to step (iii), and from step (iii) to step (iv). Preferably, the change of the medium includes a step of washing the cells comprised in the closed bioreactor system. The medium change preferably is a complete or a partial medium change.

The change of medium from step (0) to step (i) preferably comprises a partial medium change, preferably wherein the medium of step (0) remains unchanged for an amount of about 5 to 75% v/v, preferably 25% v/v. Step (0), step (ii), step (ii) (a), step (iii) and step (iv) preferably are conducted comprising perfusion of the medium.

Cell aggregates formed during step (0) preferably are dissociated within the closed bioreactor system.

The differentiated cells preferably are selected from the group consisting of cardiomyocytes, skeletal muscle cells, fibroblasts, stromal cells, endothelial cells, and leukocytes.

The present invention further relates to a population of differentiated cells obtainable by the method of the invention.

The present invention further relates to a population of differentiated cells obtained by the method of the invention.

### BRIEF DESCRIPTION OF THE DRAWINGS

The invention will be better understood with reference to the detailed description when considered in conjunction with the non-limiting examples and the accompanying drawings, in which:

FIG. 1 shows the establishment of a medium exchange strategy. FIG. 1A shows: Morphology of iPSC aggregates. Depicted are images of wells of a 24-well plate containing aggregates that were sampled from ambr15 suspension cultures. The images were acquired using a Cellavista Cell Imager. Scale bars: 3 mm. FIG. 1B shows: iPSC expansion rates after four days of culture. Mean±SD. C: Expression of pluripotency-related markers in iPSCs after four days of culture. Single values represent one bioreactor.

FIG. 2 shows the morphology of iPSCs during establishment of expansion strategy. IPSCs were transferred from static cell culture (d0) to suspension culture (d1-4). On day 4, aggregates were dissociated into smaller clumps (3-8 min).

FIG. 3 shows an exemplary overview of an iPSC suspension culture strategy corresponding to the optional step (0) of the method of the invention.

FIG. 4 shows the long-term culture of iPSCs in suspension. FIG. 4A shows: Expansion rate at the end of the individual passages. Mean of vessels±SD. FIG. 4B shows: Aggregate sizes at the end of individual passages. Mean of vessels±SD. FIG. 4C shows: Calculated accumulated expansion rate during culture. FIG. 4D shows: Expression of pluripotency-related genes at the end of individual passages during Cultivation Optimization Run 12. Results are representative for Cultivation Optimization Run 13.

FIG. 5 shows the cardiac differentiation of iPSCs cultivated with the developed suspension culture strategy. FIG. 5A shows the differentiation of iPSCs of an early passage (Cultivation Optimization Run 12) and a late passage (Cultivation Optimization Run 13). Passage 0 iPSCs were treated with 6 μM CHIR and IWP4 was added on day 2. Passage 8 iPSCs were treated with 18 μM CHIR and IWP4 was added on day2. FIG. 5B shows the differentiation of iPSCs of passage 8. Differentiation efficiency is highly dependent of CHIR concentration and the time point of IWP4 addition.

FIG. 6 shows iPSC suspension culture in the UniVessel 0.5L and 2L system. FIG. 6A shows: Aggregate sizes during passage 0. FIG. 6B shows: Inline permittivity measurements during passage 0 of UniVessel Proof of Concept run 05 using the BioPAT Viamass probe compared to cell concentration measured with the Nucleocounter-200. FIG. 6C shows: Expression of pluripotency-related genes in iPSCs cultivated in the UniVessel 0.5L. FIG. 6D shows: Expression of pluripotency-related genes in iPSCs cultivated in the UniVessel 2L.

FIG. 7 shows the morphology of iPSC aggregates during long-term culture in the UniVessel 0.5L system. Depicted are iPSCs at the end of each passage.

FIG. 8 shows the Optimization of iPSC suspension culture in the UniVessel 0.5L system. FIG. 8A shows: Aggregate size during long-term cultivation. FIG. 8B shows: Expression of pluripotency-related genes at the end of each passage. FIG. 8C shows: Inline permittivity measurements during long-term culture using the BioPAT Viamass probe compared to cell concentration measured with the Nucleocounter-200.

FIG. 9 shows Scale up of cardiac differentiation in the UniVessel system. FIG. 9A shows: Expression of cardiac markers during cardiac differentiation. FIG. 9B shows: Cell concentration during cardiac differentiation. FIG. 9C shows: Morphology of iPSC-CM aggregates. FIG. 9D shows: Morphology of singularized iPSC-CMs three days after plating.

FIG. 10 shows the morphology of aggregates and cell during the culture. FIG. 10A shows: iPSCs on day 4 of passage 0. FIG. 10B shows: iPSCs on day 4 of passage 1. FIG. 10C shows: iPSC-CMs on day 21 of differentiation (day of harvest). FIG. 10D shows: singularized iPSC-CM one day after plating.

### DETAILED DESCRIPTION OF THE INVENTION

The present invention is described in detail in the following and will also be further illustrated by the appended examples and figures.

In the present invention it was successfully shown that pluripotent stem cells (PSC) can be surprisingly directly differentiated in a closed bioreactor system (see Examples 6 and 7), thereby allowing the production of differentiated cells in large quantities in comparison to differentiation in flasks or dishes. The invention therefore allows for the automated differentiation of PSCs in a closed system and thus reduces the number of manual operations such as the transfer of the PSC. The method of the present invention thus is easier, faster and less expensive than conventional culture systems and allows further automatization of PSC differentiation. The method of the invention can be further complemented by a novel and inventive step of dissociating cell aggregates formed during differentiation (see Example 8). Since the method of the invention can, as mentioned above, be carried out in a closed system, it has the further advantage that is ideally suited for establishing a GMP compliant manufacturing process for differentiated cells.

Accordingly, the present invention relates to a method of producing a population of differentiated cells from pluripotent stem cells (PSC) in suspension culture in a bioreactor, the method comprising
(i) cultivating the PSC under suitable conditions to allow mesodermal induction,
(ii) inducing differentiation under suitable conditions of the PSC of step (i), and
(iii) optionally enriching the PSC of step (ii),
   wherein steps (i) to (iii) are conducted in a closed bioreactor system, thereby producing a population of differentiated cells.

Thus, the method of the invention generally describes a 3-step process including first mesodermal induction, following by inducing differentiation or specification and an optional final selection of the PSC, or to be more specific, of the differentiated cells produced by the method of the invention. All of the steps of the method of the invention described herein generally are carried out in the closed bioreactor system unless stated otherwise.

Step (i) of the method of the invention requires cultivating the PSC under suitable conditions to allow mesodermal induction. “Conditions to allow mesodermal induction” are known in prior art. Illustrative examples for the conditions to allow mesodermal induction are, e.g., described in WO2015/040142 and Saad et al. (2021), ChemMedChem 16 (21): 3300-3305, hereby incorporated by reference in its entirety. In an exemplary embodiment, which can be used, e.g., in the differentiation to cardiomyocytes and/or stromal cells, these conditions include a medium such as RPMI-1640, comprising about 0.1-10% B27 or B27 minus insulin, preferably 0.5-8%, more preferably 1-6%, even more preferably 1.-5%, even more preferably 1.5-4%, and most preferably about 2% B27 or B27 minus insulin; 10-1000 μM ascorbic acid, preferably 50-400 μM, more preferably 100-300 μM, even more preferably 150-250 μM, and most preferably about 200 μM of ascorbic acid or a salt or a derivative thereof; about 0.1 to 10 mM sodium pyruvate, preferably about 1 mM sodium pyruvate; 1-20 ng/ml Activin A, preferably 2.5-18 ng/ml, more preferably 5-16 ng/ml, even more preferably 7.5-14 ng/ml, still more preferably 8-12 ng/ml, most preferably 8.5-10 ng/ml, and even most preferably about 9 ng/ml Activin A; 1-20 ng/ml BMP4, preferably 2-15 ng/ml, more preferably 2.5-10 ng/ml, more preferably 3-8 ng/ml, most preferably 4-6 ng/ml, and even most preferably about 5 ng/ml BMP4; 0.1-10 ng/ml bFGF, preferably 1-9 ng/ml, more preferably 2-8 ng/ml, even more preferably 3-7 ng/ml, most preferably 4-6 ng/ml, and even most preferably about 5 ng/ml bFGF; and optionally about 0.1-10 μM CHIR99021, preferably 0.2-9 μM, more preferably 0.3-8 μM, even more preferably 0.4-7 μM, still more preferably 0.5-6 μM, more preferably 1-5 μM, more preferably 1-4 μM, more preferably 1-3 μM, most preferably about 2 μM CHIR99021. This medium may be termed “mesoderm induction medium (MIM)”. This medium can initially, e.g., during the first 12 to 36 h, preferably 18 to 30 h, more preferably 22 to 26 h and most preferably the first 24 h of step (i) of the method of the invention additionally comprise 5 to 75% v/v iPS-brew. More preferably, this medium can initially, e.g., during the first 12 to 36 h, preferably 18 to 30 h, more preferably 22 to 26 h and most preferably the first 24 h additionally comprise 10 to 50% v/v iPS-brew. More preferably, this medium can initially, e.g., during the first 12 to 36 h, preferably 18 to 30 h, more preferably 22 to 26 h and most preferably the first 24 h of step (i) of the method of the invention additionally comprise 15 to 35% v/v iPS-brew. More preferably, this medium can initially, e.g., during the first 12 to 36 h, preferably 18 to 30 h, more preferably 22 to 26 h and most preferably the first 24 h of step (i) of the method of the invention additionally comprise 20 to 30% v/v iPS-brew. More preferably, this medium can initially, e.g., during the first 12 to 36 h, preferably 18 to 30 h, more preferably 22 to 26 h and most preferably the first 24 h of step (i) of the method of the invention additionally comprise about 25% v/v iPS-brew.

Cell types and/or tissues that originate from mesoderm include muscle (smooth, cardiac and skeletal), the muscles of the tongue (occipital somites), the pharyngeal arches muscle (muscles of mastication, muscles of facial expressions), connective tissue, dermis and subcutaneous layer of the skin, bone and cartilage, dura mater, endothelium of blood vessels, red blood cells, white blood cells, and microglia, Dentine of teeth, the kidneys and the adrenal cortex. In line with this, the differentiated cells preferably are selected from the group consisting of cardiomyocytes, skeletal muscle cells, fibroblasts, stromal cells, endothelial cells, leukocytes, and myocytes. The cell types originating from mesoderm can thus be the differentiated cells obtained by the method of the invention. More preferably, the differentiated cells are cardiomyocytes. More preferably, the differentiated cells are fibroblasts. More preferably, the differentiated cells are stromal cells. More preferably, the differentiated cells are endothelial cells. More preferably, the differentiated cells are leukocytes. More preferably, the differentiated cells are myocytes. More preferably, the differentiated cells are skeletal muscle cells. Accordingly, “mesodermal induction” as used herein relates to conditions, which lead to formation of (precursors) of cell types and/or tissues that originate from mesoderm.

Step (i) of the method of the invention is preferably conducted for about 24 hours to 5 days, for about 2 to 4 days, for about 2.5 to 3.5 days, or more preferably for about 3 days.

Step (ii) of the method of the invention requires inducing differentiation under suitable conditions of the PSC of step (i). In this step, specification to a cell types and/or tissues that originate from mesoderm can be achieved, e.g., specification to cardiomyocytes. An exemplary description of this step for specification towards cardiomyocytes is, e.g., described in WO2015/040142 and Saad et al. (2021), ChemMedChem 16 (21): 3300-3305, hereby incorporated by reference in its entirety. “Inducing differentiation under suitable conditions of the PSC of step (i)” may thus include culturing the PSC obtained from step (i) in a medium, e.g., a cardiomyocyte induction medium (CM-IM). The CM-IM may be a medium such as RPMI-1640, which may comprise about 0.1-10% B27® or B27® minus insulin, preferably 0.5-8%, more preferably 1-6%, even more preferably 1.5-4%, and most preferably about 2% B27® or B27® minus insulin; 10-1000 μM, preferably 50-400 μM, more preferably 100-300 μM, even more preferably 150-250 μM, and most preferably about 200 μM of ascorbic acid or a salt or a derivative thereof; about 0.1 to 10 mM sodium pyruvate, preferably about 1 mM sodium pyruvate; and about 1-15 μM IWP4, preferably 1-9 μM, more preferably 2-8 M, even more preferably 3-7 μM, still more preferably 4-6 μM, and most preferably about 5 μM IWP4. The ascorbic acid may be delivered in the free form or as a salt. Since ascorbate is the active ingredient, any salt or derivative of ascorbic acid may be used, which provides the ascorbate to the cells, provided the counter ion has no detrimental effect on the cells. As shown in the examples, one suitable salt or derivative of ascorbic acid is ascorbate-2-phosphate, preferably L-ascorbic acid-2-phosphate sesquimagnesium salt hydrate.

The method of the invention is also suitable to produce skeletal muscle cells (as population of differentiated cells). Methods for producing muscle tissue are, e.g., disclosed in WO 2021/074126, hereby incorporated by reference in its entirety. In particular, engineered skeletal muscle tissue in an exemplary embodiment can be produced from pluripotent stem cells as follows: (i) inducing mesoderm differentiation of the pluripotent stem cells by culturing pluripotent stem cells in a basal medium comprising an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof (corresponding to step (a) of the method of the invention). This can be followed by (ii) inducing myogenic specification by culturing the cells obtained in step (i) in a basal medium comprising an effective amount of (a) a gamma-secretase/NOTCH inhibitor, (b) FGF2, and (c) a serum-free additive as in (i), followed by continuing culturing in said medium with the addition of an effective amount of (d) HGF, followed by culturing said cells in a basal medium comprising an effective amount of (a) a gamma secretase/NOTCH inhibitor, (b) HGF, (c) a serum-free additive as in (i), and (d) knockout serum replacement (KSR) (corresponding to step (b) of the method of the invention). Optionally, the cells can be expanded and matured into skeletal myoblasts and satellite cells by culturing the cells obtained in step (ii) in a basal medium comprising an effective amount of (a) HGF, (b) a serum-free additive as in (i), and (c) knockout serum replacement (KSR); and optionally maturing the cells into skeletal myotubes and satellite cells after harvesting by culturing the cells obtained in step (iii) dispersed in an extracellular matrix under mechanical stimulation in a basal medium, comprising an effective amount of (a) a serum-free additive as in step (i) and (b) an additional serum-free additive comprising albumin, transferrin, ethanolamine, selenium or a bioavailable salt thereof, L-carnitine, fatty acid additive, and triodo-L-thyronine (T3); thereby producing artificial skeletal muscle tissue. This final step (iii) can be performed outside the closed bioreactor system.

Methods for producing fibroblasts are known to a person skilled in the art. Exemplary methods are, e.g., described in Shamis et al. (2013), PLOS ONE 8 (12): e83755, which is hereby incorporated by reference. Methods for producing stromal cells are known to a person skilled in the art. Exemplary methods are, e.g., described in WO 2022/023451 A1 or Santos et al. (2021), J. Vis. Exp., 174: e62700, which are hereby incorporated by reference in its entirety. Methods for producing endothelial cells are known to a person skilled in the art. Exemplary methods are, e.g., described in Jang et al. (2019), Am J Pathol., 189 (3): 502-512. Methods for producing leukocytes are known to a person skilled in the art.

Step (ii) of the method of the invention is preferably conducted for about 4 to 10 days, for about 5 to 9 days, for about 6 to 8 days, or more preferably for about 7 days.

The method of the invention may include a step of enriching or purifying the differentiated cells, i.e. the PSC of step (ii), in the closed bioreactor system. “Enriching” or “purifying” as used herein relates to a relative increase of the number of the differentiated cells in comparison to unwanted cells. Enriching or purifying of the differentiated cells can be achieved by metabolic selection, or with a selectable marker.

In one embodiment, the step of enriching the differentiated cells requires a metabolic selection. “Metabolic selection” as used herein relates to a process, which selects a certain subtype of a population of cells based on their metabolic specifics. E.g., cardiomyocytes may survive under glucose-starvation as long as they are supplied with L-Lactate (see, e.g., Tohyama et al. (2013), Cell Stem Cell 12:127-137). An exemplary metabolic selection is also described in WO 2007/088874 A1, both of which are hereby incorporated by reference. In order to enrich a population of cells comprising cardiomyocytes with cardiomyocytes, the population of cells can be conducted to metabolic selection, i.e. be cultured in a glucose-free medium, which has been supplemented with lactate. Preferred L-lactate concentrations include 1 to 5 mM lactate, more preferably 1 to 4 mM lactate, more preferably 2 to 4 mM lactate, more preferably 2 to 3 mM lactate and most preferably about 2.8 mM lactate. A preferred selection medium is RPMI-1640 without glucose supplemented with 1 to 5 mM lactate, more preferably 1 to 4 mM lactate, more preferably 2 to 4 mM lactate, more preferably 2 to 3 mM lactate and most preferably about 2.8 mM lactate, 0.01 to 1 mM 2-mercaptoethanol, more preferably 0.05 to 0.5 mM 2-mercaptoethanol, more preferably 0.075 to 0.2 mM 2-mercaptoethanol, most preferably about 0.1 mM 2-mercaptothanol and a buffer such as HEPES, e.g., about 2 to 8 mM HEPES, more preferably 3 to 6 mM HEPES and most preferably about 4.5 mM HEPES.

Optional step (iii) of the method of the invention, in particular the metabolic selection, is preferably conducted for about 5 to 10 days, for about 6 to 9 days, for about 6 to 8 days, or more preferably for about 7 days.

A “selectable marker” is a gene introduced into a cell that confers a trait suitable for artificial selection. Selection by selectable markers and selectable markers are known to a person skilled in the art. Within the context of the invention, the selectable marker preferably is under the control of a promoter, which is only expressed in the differentiated cells and preferably not expressed in any other unwanted differentiated cell type. Selectable markers include, but are not limited to, antibiotic resistance genes such as the Neo gene from Tn5, which confers resistance to kanamycin and geneticin.

After the optional selection step (iii), the cells resulting from step (iii) may be recovered. Accordingly, after step (iii) of the method of the invention a further step (iv) can be conducted of recovery of the PSC of step (iii) under suitable conditions in the closed bioreactor system. Recovery can be carried out in a basal serum-free medium (BSFM). BSFM preferably is RPMI-1640 supplemented with about 0.1-10% B27® or B27® minus insulin, preferably 0.5-8%, more preferably 1-6%, even more preferably 1.5-4%, and most preferably about 2% B27® or B27® minus insulin; 10-1000 M, preferably 50-400 μM, more preferably 100-300 μM, even more preferably 150-250 μM, and most preferably about 200 μM of ascorbic acid or a salt or a derivative thereof; and about 0.1 to 10 mM sodium pyruvate. Optional step (iv) of the method of the invention is preferably conducted for about 12 hours to 5 days, for about 1 to 3 days, or preferably for about 2 days.

Between steps (ii) and (iii) of the method of the invention a further recovery step may be carried out, which can also allow further expansion of the cells. Accordingly, after step (ii) a further step (ii) (a) may be conducted of expanding the PSC of step (ii) under suitable conditions. Suitable conditions may include recovery in a BSFM described herein.

Step (ii) (a) of the method of the invention is preferably conducted for about 1 to 5 days, for about 1 to 3 days, or preferably for about 2 days.

After the differentiation step (ii), the optional metabolic selection step (iii) or the optional recovery step (iv) of the method of the invention, the population of differentiated cells may be harvested. Accordingly, after step (ii) of the method of the invention a further step (v) may be conducted of harvesting of the population of differentiated cells. Accordingly, after optional step (iii) of the method of the invention a further step (v) may be conducted of harvesting of the population of differentiated cells. Accordingly, after optional step (iv) of the method of the invention a further step (v) may be conducted of harvesting of the population of differentiated cells. Methods for harvesting cells are known to a person skilled in the art and may include centrifuging the cells to separate them from the culture medium.

The cells cultured in the closed bioreactor system while carrying out the method of the invention may form aggregates. The method of the invention thus can be complemented by a further step of dissociating such cell aggregates comprised in the population of differentiated cells obtained by the method of the invention directly in the closed bioreactor system. As shown in Example 8, such a step of dissociating cell aggregates is highly suitable for the harvest of iPSC-derived differentiated cells such as cardiomyocytes produced in bioreactors and provides high quality differentiated cells. Accordingly, the method of the invention may comprise a step of dissociating aggregates formed during any one of steps (i) to (iv). Harvesting of the population of differentiated cells (step (v)) may include dissociating aggregates formed during any one of steps (i) to (iv).

In one particular embodiment, dissociating aggregates includes:
(a) allowing the cells or aggregates to sediment at the bottom of the closed bioreactor system and removing the supernatant;
(b) adding a cell dissociation agent, preferably an enzyme such as trypsin;
(c) agitating the cells or cell aggregates;
(d) repeating steps (a)-(c) three times; and
(e) stopping the cell dissociation by adding stop medium, preferably wherein the stop medium comprises Knockout-Serum replacement,
   preferably wherein the differentiated cells are cardiomyocytes.

Steps (a)-(c) can be repeated (step (d)). Steps (a)-(c) can be repeated once (step (d)). Steps (a)-(c) can be repeated twice (step (d)). Steps (a)-(c) can be repeated three times (step (d)). Steps (a)-(c) can be repeated four times (step (d)). They can however also only be carried out once (no step (d)).

In one particular embodiment, dissociating aggregates includes:
(a) allowing the cells or aggregates to sediment at the bottom of the closed bioreactor system and removing the supernatant;
(b) adding a cell dissociation agent, preferably an enzyme such as trypsin;
(c) agitating the cells or cell aggregates;
(d) repeating steps (a)-(c) two times; and
(e) stopping the cell dissociation by adding stop medium, preferably wherein the stop medium comprises Knockout-Serum replacement,
   preferably wherein the differentiated cells are cardiomyocytes.

In one particular embodiment, dissociating aggregates includes:
(a) allowing the cells or aggregates to sediment at the bottom of the closed bioreactor system and removing the supernatant;
(b) adding a cell dissociation agent, preferably an enzyme such as trypsin;
(c) agitating the cells or cell aggregates;
(d) repeating steps (a)-(c) once; and
(e) stopping the cell dissociation by adding stop medium, preferably wherein the stop medium comprises Knockout-Serum replacement,
   preferably wherein the differentiated cells are cardiomyocytes.

In one particular embodiment, dissociating aggregates includes:
(a) allowing the cells or aggregates to sediment at the bottom of the closed bioreactor system and removing the supernatant;
(b) adding a cell dissociation agent, preferably an enzyme such as trypsin;
(c) agitating the cells or cell aggregates; and
(e) stopping the cell dissociation by adding stop medium, preferably wherein the stop medium comprises Knockout-Serum replacement,
   preferably wherein the differentiated cells are cardiomyocytes.

Allowing the cells or aggregates to sediment at the bottom of the closed bioreactor system in step (a) may include stopping the agitation of the cells comprised in the closed bioreactor system. This may allow the cells to sediment by gravity, thereby forming a supernatant. The duration of the sedimentation step depends on the volume, cell density and the closed bioreactor system. However, a person skilled in the art is capable to determine which duration is sufficient by determining the amount of cells or cell aggregates present in the supernatant, which has been removed. In one embodiment, the sedimentation step is carried out for about 1 to 10 min, for about 2 to 8 min, for about 3 to 7 min, for about 4 to 6 min or preferably for about 5 min.

After the cell or cell aggregates have sedimented at the bottom of the closed bioreactor system, the supernatant is removed (step (a)) and replaced with a cell dissociation reagent or a solution comprising the cell dissociation reagent. The cell dissociation reagent preferably is an enzyme such as trypsin, more preferably the commercially available TrypLE™ Select obtainable from ThermoFisher Scientific. The concentration of the dissociation reagent is dependent on the cell or cell aggregates, temperature, agitation, the closed bioreactor system and the like. However, it is within the abilities of a person skilled in the art to optimize the concentration. After addition of the cell dissociation reagent to the closed bioreactor system and the cells or cell aggregates are agitated. This step ensures an even distribution of the cell dissociation reagent in the medium comprising the cells or cell aggregates. The cells or cell aggregates may be agitated within the closed bioreactor system for about 5 to 15 s, preferably for about 10 s.

The cells or cell aggregates are incubated together with the cell dissociation reagent for a sufficient amount of time to dissociate the cell aggregates. Since the incubation step is identical with step (a), the disclosure relating to step (a) also apply to the incubation step. The incubation step preferably is carried out for a time sufficient to achieve dissociated cell aggregates.

The last iteration of step (d), i.e. steps (a)-(c), may include an elongated agitation step (c). Elongated refers to a period of time, which is longer than the period of time, for which the cells are sedimented and/or incubated with the cell dissociation reagent (step (a)). Said elongated agitation may refer to an agitation for about 15 to 75 min, for about 30 to 60 min, for about 40 to 50 min or preferably for about 45 min.

After the final agitation step, the cell dissociation is to be stopped by addition of a stop medium. The ingredients of the stop medium depend on the cell dissociation reagent. In one embodiment, e.g., if the cell dissociation reagent is a chelating agent, the cell dissociation can be stopped by adding an excess volume of culture medium. In another embodiment, in particular in connection with the cell dissociation reagent being an enzyme such as a proteolytic enzyme, the cell dissociation is stopped by adding an excess volume of culture medium supplement with serum proteins, e.g., as comprised in fetal calf serum, human platelet lysate (hPL), serum albumin such as bovine serum albumin (BSA), Gibco™ KnockOut SR-Defined, FBS-free Formulation, preferably Gibco™ KnockOut SR-Defined, FBS-free Formulation.

The present invention further relates to a method of dissociating cell aggregates in a closed bioreactor system, the method comprising:
(a) allowing the cells or aggregates to sediment at the bottom of the closed bioreactor system and removing the supernatant;
(b) adding a cell dissociation agent, preferably an enzyme such as trypsin;
(c) agitating the cells or cell aggregates;
(d) repeating steps (a)-(c) three times; and
(e) stopping the cell dissociation by adding stop medium, preferably wherein the stop medium comprises Knockout-Serum replacement,
   preferably wherein the differentiated cells are cardiomyocytes. Steps (a)-(c) can be repeated (step (d)). Steps (a)-(c) can be repeated once (step (d)). Steps (a)-(c) can be repeated twice (step (d)). Steps (a)-(c) can be repeated three times (step (d)). Steps (a)-(c) can be repeated four times (step (d)). They can however also only be carried out once (no step (d)).

The PSC needed for the method of the invention can be expanded in the same closed bioreactor system, in which the later differentiation (steps (i) to (iii) of the method of the invention) takes place. Methods for the expansion of PSC are known to a person skilled in the art and are, e.g., described in WO 2021/116362 or WO 2021/116361, both of which are incorporated by reference in its entirety.

Accordingly, optional step (0) of the method of the invention may comprise the following steps (i) adding an inhibitor of ROCK (ROCKi) to pluripotent stem cells being cultivated in suspension in the bioreactor; (ii) adding a cell dissociation agent, thereby dissociating aggregates of the pluripotent stem cells; (iii) diluting the cell dissociation agent added in step (ii) by adding an excess volume of culture medium sufficient to decrease the concentration of the cell dissociation agent to a concentration at which cell aggregates can form again; and (iv) culturing of the mixture obtained in step (iii) under suitable conditions that allow the expansion of the PSCs.

The PSCs cultured in suspension in the bioreactor in the optional step (0) of the method of the invention are preferably cultured in a culture medium. Culture media that allow the expansion of the PSCs are known to a person skilled in the art and include, but are not limited to, IPS-Brew, iPS-Brew XF, E8, StemFlex, mTeSR1, PluriSTEM, StemMACS, TeSRTM2, Corning NutriStem hPSC XF Medium, Essential 8 Medium (ThermoFisher Scientific), StemFit Basic02 (Ajinomoto Co. Inc), to name only a few. In one illustrative example, the culture medium is IPS-Brew that is available in GMP grade from Miltenyi Biotec, Germany. IPS-Brew preferably is supplement with iPS-brew supplement also available from Miltenyi Biotec, Germany.

Cell aggregates can particularly be formed in the optional initial PSC expansion step (0) of the method of the invention. During expansion, the PSC form cell aggregates, which increase in size over time. When they reach a critical diameter, the supply with nutrients, oxygen and the like but also the removal of (toxic) metabolic end products is hindered more and more in the inner section. Thus, it might be necessary to passage the PSC during expansion, or in other words, to dissociate cell aggregates grown in optional step (0) of the method of the invention. Methods for dissociating cell aggregates in a closed bioreactor system, i.e. without the need to remove the cells during the dissociation of the cell aggregates/passaging, are, e.g., described in WO 2021/116362. Accordingly, cell aggregates formed during step (0) preferably are dissociated within the closed bioreactor system

As used herein, the terms “aggregate” and “cell aggregate”, which may be used interchangeably, refer to a plurality of (induced) pluripotent stem cells in which an association between the cells is caused by cell-cell interaction (e.g., by biologic attachments to one another). Biological attachment may be, for example, through surface proteins, such integrins, immunoglobulins, cadherins, selectins, or other cell adhesion molecules. For example, cells may spontaneously associate in suspension and form cell-cell attachments (e.g., self-assembly), thereby forming aggregates of the PSCs. In some embodiments, a cell aggregate may be substantially homogeneous (i.e., mostly containing cells of the same type). In some embodiments, a cell aggregate may be heterogeneous, (i.e., containing cells of more than one type).

In some embodiments, the aggregates have an average diameter of between about 150 and 800 μm in size prior to dissociation. In some embodiments, the aggregates have an average diameter of at least about 800 μm in size in step (0) of the method of the invention prior to dissociation. In some embodiments, the aggregates have an average diameter of at least about 600 μm in size prior to dissociation. In some embodiments, the aggregates have an average diameter of at least about 500 μm in size prior to dissociation. In some embodiments, the aggregates have an average diameter of at least about 400 μm in size prior to dissociation. In some embodiments, the aggregates have an average diameter of at least about 300 μm in size prior to dissociation. In some embodiments, the aggregates have an average diameter of at least about 200 μm in size prior to dissociation. In some embodiments, the aggregates have an average diameter of at least about 150 μm in size prior to dissociation. In a preferred embodiment, the aggregates have an average diameter of between about 300 and 500 μm in size prior to dissociation. In a preferred embodiment, the aggregates have an average diameter of between about 150 and 300 μm in size prior to dissociation.

The formation of extensive aggregate dimensions is preferably avoided since diameters exceeding about 300 μm may result in cell necrosis due to the limited nutrient and gas diffusion into the tissue/aggregate center. Eventually, uncontrolled differentiation—particularly in large PSC aggregates—might also occur. The regular dissociation of aggregates into single cells at every passage is therefore important. As shown in the Examples, the method of the present invention solves this problem in a convenient manner. An average diameter of about 180 to 250 μm before the cell aggregate dissociation, preferably 200 to 250 μm, ideally about 200 μm can be seen the best compromise between pluripotency (in particular for PSC) and yield of cells. Accordingly, the aggregates preferably have a diameter of about 180 to 250 μm, more preferably about 200 to 250 μm and most preferably of about 200 μm in size in step (0) of the method of the invention prior to dissociation.

As used herein, the terms “dissociate” and “dissociation” refer to a process of separating aggregated cells from one another. For example, during dissociation, the cell-cell interaction between cells and between cells may be disrupted, thereby breaking apart the cells in the aggregate.

As used herein, the term “cell dissociation agent” or “cell dissociation reagent”-both of which can be used interchangeably-refer to a reagent or a solution comprising one or more reagents that separate cells from one another, such as, for example, chelating agent(s). For example, a dissociation reagent may break the bonds between cells, thereby disrupting the aggregation of cells in suspension. For example, the dissociation reagent may be a chelating agent, which may cause sequestration of a molecule to weaken or break bond formation between cell adhesion proteins, e.g. by chelation to disrupt calcium- or magnesium-dependent adhesion molecules.

Accordingly, the dissociation reagent preferably is a chelating agent. A “chelating reagent” as used herein may be a (organic) compound, peptide or protein that chelates divalent cations such as Ca²⁺ or Mg²⁺. Chelation is a type of bonding of ions and molecules to metal ions. It involves the formation or presence of two or more separate coordinate bonds between a polydentate (multiple bonded) ligand and a single central atom.

The chelating agent may be selected from the group consisting of ethylenediaminetetraacetate (EDTA), ethylene glycol-bis (β-aminoethyl ether)-N,N,N′,N′-tetraacetic acid (EGTA), iminodisuccinic acid (IDS), polyaspartic acid, ethylenediamine-N,N′-disuccinic acid (EDDS), citrate, citric acid, 1,2-bis(o-aminophenoxy) ethane-N,N,N′,N′-tetraacetic acid (BAPTA), and methylglycinediacetic acid (MGDA). The chelating agent may be ethylenediaminetetraacetate (EDTA). The chelating agent may be ethylene glycol-bis (β-aminoethyl ether)-N, N,N′, N′-tetraacetic acid (EGTA). The chelating agent may be iminodisuccinic acid (IDS). The chelating agent may be polyaspartic acid. The chelating agent may be ethylenediamine-N, N′-disuccinic acid (EDDS). The chelating agent may be citrate. The chelating acid may be citric acid. The chelating agent may be 1,2-bis(o-aminophenoxy) ethane-N,N,N′, N′-tetraacetic acid (BAPTA). The chelating agent may be methylglycinediacetic acid (MGDA). Preferably, the chelating agent is EDTA. The commercially available “Versene” solution comprising EDTA available from ThermoFisher Scientific is an exemplary and preferred dissociation reagent.

The final concentration of the chelating agent may be at least 100 μM, in a range of about 100 to about 1000 μM, in a range of about 250 to about 750 μM, in a range of about 400 to about 600 μM or is about 500 μM, preferably about 500 μM. The final concentration of the chelating agent that is used in step (ii) may be at least 100 μM EDTA, in a range of about 100 to about 1000 μM EDTA, in a range of about 250 to about 750 μM EDTA, in a range of about 400 to about 600 μM EDTA or is about 500 μM EDTA, preferably about 500 μM EDTA.

The cell dissociation agent preferably is essentially free of enzymes such as proteolytic enzymes, particularly in step (0). “Essentially free of enzymes” in this context can relate to a cell dissociation agent, to which no enzymes, preferably proteolytic enzymes such as trypsin, pepsin, collagenases such as collagenase I, collagenase II, or collagenase B etc. have been added. Thus, “essentially free of enzymes” may exclude enzymes or solutions comprising enzymes including Accutase, Accumax, trypsin, TrypLE Select and collagenase B. However, the cell dissociation may also be an enzyme such as proteolytic enzymes, particularly in step (v). Exemplary enzymes, preferably proteolytic enzymes such as trypsin, pepsin, collagenases such as collagenase I, collagenase II, or collagenase B, preferably trypsin, more preferably TrypLE Select. Commercially available enzymes or solutions comprising enzymes including Accutase, Accumax, trypsin, TrypLE Select and collagenase B.

As used herein, the terms “dissociated” and “dissociated aggregate” refer to single cells, or cell aggregates or clusters that are smaller than the original cell aggregates (i.e., smaller than a pre-dissociation aggregate, e.g. as in step (i) of step (0) or step (v) of the method of the invention). For example, a dissociated aggregate may comprise about 50% or less surface area, volume, or diameter relative to a pre-dissociation cell aggregate. The dissociated aggregate may consist of cell aggregates having 2 to 10 cells/PSCs or having 1 to 10 cells/PSCs. Preferably, the dissociated cell aggregates have a diameter of about 25 μm to about 130 μm, more preferably of about 80 μm to about 100 μm after dissociation.

The size of the resulting dissociated aggregates may be controlled by the amount of time, for which the cell dissociation reagent in step (ii) of step (0) of the method of the invention is undiluted. Accordingly, the aggregates preferably are dissociated in step (ii) of step (0) for at least about 1 min, at least about 2 min, at least about 3 min, at least about 5 min, at least about 10 min, for 1 to 20 min, for about 10 to about 20 min, for about 10 to about 15 min or for up to about 15 min, preferably for about 15 min.

The dilution step (iii) of optional step (0) or optional step (e) of step (v) of the method of the invention decreases the concentration of the cell dissociation agent to a concentration at which cell aggregates can form again, thereby stopping the cell dissociation reaction. In case the cell dissociation agent is a chelating agent, the excess volume of the added medium in optional step (iii) of step (0) or optional step (e) of step (v) of the method of the invention can provide a sufficient amount of ions to saturate the chelating agent so that the ions of the added culture medium can replace the ions bound by the chelating agent in optional step (iii) of step (0) or optional step (e) of step (v) of the method of the invention. If EDTA is used as chelating agent, preferably with a (final) concentration of about 500 μM, the dissociation reagent added in optional step (iii) of step (0) or optional step (e) of step (v) of the method of the invention can be diluted by an excess of 5 volumes of culture medium. Preferably, the concentration of the dissociation agent after dilution in optional step (iii) of step (0) or optional step (e) of step (v) in the resulting mixture is about 100 μM or less, about 95 μM or less, about 90 μM or less, about 80 μM or less, about 70 μM or less, in a range of about 100 to about 1 μM, or in a range of about 90 to about 1 μM. In case the dissociation reagent is EDTA, the concentration of the dissociation agent after dilution in optional step (iii) of step (0) or optional step (e) of step (v) of the method of the invention in the resulting mixture is about 100 μM or less EDTA, about 95 μM or less EDTA, about 90 μM or less EDTA, about 80 μM or less EDTA, about 70 μM or less EDTA, in a range of about 100 to about 1 μM EDTA, or in a range of about 90 to about 1 μM EDTA.

“Excess volume” as used herein may relate to a volume that exceeds the amount of dissociation reagent added in optional step (iii) of step (0) or optional step (e) of step (v) of the method of the invention by at least 2-fold, at least 3-fold, at least 4-fold, at least 5-fold, at least 7.5-fold, at least 10-fold, at least 20-fold or at least 30-fold.

Inhibitors of ROCK (ROCKi) are well known to a person skilled in the art. Examples of ROCKi include, but are not limited to, AS1892802, fasudil hydrochloride, GSK 269962, GSK 429286, H 1152, HA 1100, OXA 06, RKI 1447, SB 772077B, SR 3677, TC-S 7001, thiazovivin and Y27632. Preferably, the ROCKi is Y27632. Preferably, the ROCKi is thiazovivin.

An inhibitor of ROCK may be added in the culture medium used in step (iii) of step (0) of the method of the invention to facilitate cell survival and cell re-aggregation of the PSCs (see e.g. Example 4). Accordingly, the culture medium in step (iii) of step (0) of the method of the invention preferably comprises a ROCKi. Similarly, a ROCKi is added in step (i) of step (0) of the method of the invention to the PSCs that are cultivated in a bioreactor. The addition of the ROCKi may be done about 2 hours to about 4 hours prior to step (ii) of step (0) of the method of the invention.

Continuous administration of a ROCKi to PSCs suspension cultures after (re-) formation of aggregates might decrease the yield of the PSC culture. Thus, in one embodiment of the invention, the culture medium is changed to a medium essentially free of a ROCKi, preferably after the PSCs have formed aggregates again. Accordingly, step (0) the method of the present invention may further comprise step (v): exchanging the medium to a medium essentially free of the ROCKi. It may take up to 3 days until aggregates of the PSCs have been formed again in the suspension culture. Accordingly, the culture medium that is used after dilution step (iii) of step (0) of the method of the invention preferably comprises a ROCKi for about 1 to about 3 days, preferably 2 days. In other words, step (iv) of step (0) of the method of the invention is performed for about 1 to 3 days, preferably about 2 days. The exchange of the medium to a medium essentially free of the ROCKi may start for about 1 to 3 days, preferably about 2 days after step (iii) of step (0) of the method of the invention, i.e. after dilution of the cell dissociation agent.

Table 1 shows an overview of exemplary different media that can be used in the method of the invention and Table 2 shows an exemplary process schedule for carrying out the method of the invention. Both tables are examples for a method of differentiating PSC to cardiomyocytes.

TABLE 1
Exemplary media useful for the method of the invention,
e.g. for the production of cardiomyocytes

| Medium | Composition |
|---|---|
| iPS-brew | iPS-Brew basal medium + 1x iPS-brew supplement |
| Basal Serum-Free | RPMI + 2% B27 + 200 μmol/L I-ascorbic acid-2-phosphate |
| Medium (BSFM) | sesquimagnesium salt hydrate + 1 mM sodium pyruvate |
| Mesodermal Induction | RPMI + 2% B27 minus insulin + 200 μmol/L I-ascorbic acid-2- |
| Medium (MIM) | phosphate sesquimagnesium salt hydrate + 1 mM sodium |
| | pyruvate + 9 ng/mL Activin A + 5 ng/mL BMP4 + 5 ng/mL bFGF + |
| | 2 μM CHIR99021 |
| BSFM + 25% iPS-brew + | BSFM + 25% iPS-brew + 9 ng/mL Activin A + 5 ng/mL BMP4 + 5 |
| ABCF | ng/mL bFGF + 2 μM CHIR99021 |
| Cardiomyocyte Induction | RPMI + 2% B27 + 200 μmol/L I-ascorbic acid-2-phosphate |
| Medium (CM-IM) | sesquimagnesium salt hydrate + 1 mM sodium pyruvate + 5 μM |
| | IWP4 |
| Selection Medium | RPMI minus glucose + 2.82 mM Lactate + 0.1 mM 2- |
| | Mercaptoethanol + 4.5 mM HEPES |

TABLE 2
Exemplary process schedule for the production of cardiomyocytes.

| Time points | Step | Medium type | Medium change | Step |
|---|---|---|---|---|
| Day −1 | iPSC expansion | iPS-brew | 60% perfusion | (0) |
| Day 0 | Mesoderm Induction | BSFM + 25% iPS-brew + ABCF | — | (i) |
| Day 1 | Mesoderm Induction | MIM | 100% | (i) |
| Day 2 | Mesoderm Induction | MIM | 100% (*) | (i) |
| Day 3 | Cardiac Specification | CM-IM | 50% (perfusion) | (ii) |
| Day 4 | Cardiac Specification | CM-IM | 50% (perfusion) | (ii) |
| Day 5 | Cardiac Specification | CM-IM | 50% (perfusion) | (ii) |
| Day 6 | Cardiac Specification | CM-IM | 50% (perfusion) | (ii) |
| Day 7 | Cardiac Specification | CM-IM | 50% (perfusion) | (ii) |
| Day 8 | Cardiac Specification | CM-IM | 50% (perfusion) | (ii) |
| Day 9 | Cardiac Specification | CM-IM | 50% (perfusion) | (ii) |
| Day 10 | Expansion | BSFM | 50% (perfusion) | (ii)(a) |
| Day 11 | Expansion | BSFM | 50% (perfusion) | (ii)(a) |
| Day 12 | Metabolic Selection | Selection Medium | 50% (perfusion) | (iii) |
| Day 13 | Metabolic Selection | Selection Medium | 50% (perfusion) | (iii) |
| Day 14 | Metabolic Selection | Selection Medium | 50% (perfusion) | (iii) |
| Day 15 | Metabolic Selection | Selection Medium | 50% (perfusion) | (iii) |
| Day 16 | Metabolic Selection | Selection Medium | 50% (perfusion) | (iii) |
| Day 17 | Metabolic Selection | Selection Medium | 50% (perfusion) | (iii) |
| Day 18 | Metabolic Selection | Selection Medium | 50% (perfusion) | (iii) |
| Day 19 | Recovery | BSFM | 50% (perfusion) | (iv) |
| Day 20 | Recovery | BSFM | 50% (perfusion) | (iv) |
| Day 21 | Harvest | — | — | (v) |

(*) Complete medium change including two washing steps performed as if the medium type was switched to another medium type. The correspondence to the steps of the method of the invention is further shown.

“B27” as used herein relates to a serum-free supplement described in Brewer et al. (1993), Journal of Neuroscience Research 35:567-576. B27 may be replaced by a custom-made supplement described in the following table:

TABLE 3
Custom-made supplement to replace B27.

| Substance | Final concentration | | 25x | | Supplier |
|---|---|---|---|---|---|
| Albumin | 5 | mg/ml | 125 | mg/ml | Sigma, A9511 |
| Transferrin | 10 | μg/ml | 250 | μg/ml | Sigma, T0665 |
| EthanolamineHCl | 2 | μg/ml | 50 | μg/ml | Sigma, E6133 |
| Sodium selenite | 0.032 | μg/ml | 0.8 | μg/ml | Sigma, S5361 |
| L-CarnitineHCl | 4 | μg/ml | 100 | μg/ml | Sigma, C0283 |
| Hydrocortisone | 1 | μg/ml | 25 | μg/ml | Sigma, H2270 |
| Fatty acid | 0.5 | μl/ml | 12.5 | μl/ml | Sigma, F7050 |
| supplement | | | | | |
| Triodo-L- | 0.004 | μg/ml | 0.1 | μg/ml | Sigma, T 6397 |
| thyronine | | | | | |

“Expanding” or “expansion of” PSCs or iPSCs as described herein describes an increase of cell number due to cell division.

The term “pluripotent stem cell” (PSC) as used herein refers to any cell that is able to differentiate into every cell type of the body. As such, pluripotent stem cells offer the unique opportunity to be differentiated into essentially any tissue or organ. Currently, the most utilized pluripotent cells are embryonic stem cells (ESC) or induced pluripotent stem cells (iPSC). Human ESC-lines were first established by Thomson and coworkers (Thomson et al. (1998), Science 282:1145-1147). Human ESC research recently enabled the development of a new technology to reprogram cells of the body into an ES-like cell. This technology was pioneered by Yamanaka and coworkers in 2006 (Takahashi & Yamanaka (2006), Cell, 126:663-676). Resulting induced pluripotent cells (iPSC) show a very similar behavior as ESC and, importantly, are also able to differentiate into every cell of the body. Another example of pluripotent stem cells that can be used in the present invention are parthenogenetic (PG) (embryonic) stem cells, which, can, for example in both mouse and human, be readily derived from blastocysts developing after in vitro activation of unfertilized oocytes (cf. in this context, for example Espejel et al, Parthenogenetic embryonic stem cells are an effective cell source for therapeutic liver repopulation, Stem Cells. 2014 July; 32 (7): 1983-1988 or Didie et al, Parthenogenetic stem cells for tissue-engineered heart repair. J Clin Invest. 2013 March; 123 (3): 1285-98. Another example of suitable pluripotent stem cells that can be used herein are nuclear transfer derived PSCs (ntPSC; cf, Kang et al, Improving Cell Survival in Injected Embryos Allows Primed Pluripotent Stem Cells to Generate Chimeric Cynomolgus Monkeys, Cell Reports Volume 25, Issue 9, 27 Nov. 2018, Pages 2563-2576) In the context of the present invention, these pluripotent stem cells are however preferably not produced using a process which involves modifying the germ line genetic identity of human beings or which involves use of a human embryo for industrial or commercial purposes. Preferably, the pluripotent stem cells are of primate origin, including, but not limited to murine, rat, feline, canine, bovine, equine, simian or human origin, and more preferably they are of human origin.

Suitable induced PSCs, can for example, be obtained from the NIH human embryonic stem cell registry, the European Bank of Induced Pluripotent Stem Cells (EBiSC), the Stem Cell Repository of the German Center for Cardiovascular Research (DZHK), or ATCC, to name only a few sources. Induced pluripotent stem cells are also available for commercial use, for example, from the NINDS Human Sequence and Cell Repository (https://stemcells.nindsgenetics.org) which is operated by the U.S. National Institute of Neurological Disorders and Stroke (NINDS) and distributes human cell resources broadly to academic and industry researchers. One illustrative example of a suitable cell line that can be used in the present invention is the cell line TC-1133, an induced (unedited) pluripotent stem cell that has been derived from a cord blood stem cell. This cell line is, e.g. directly available from NINDS, USA. Preferably, TC-1133 is GMP-compliant. Further exemplary iPSC cell lines that can be used in the present invention, include but are not limited to, the Human Episomal iPSC Line of Gibco™ (order number A18945, Thermo Fisher Scientific), or the iPSC cell lines ATCC ACS-1004, ATCC ACS-1021, ATCC ACS-1025, ATCC ACS-1027 or ATCC ACS-1030 available from ATTC. Alternatively, any person skilled in the art of reprogramming can easily generate suitable iPSC lines by known protocols such as the one described by Okita et al, “A more efficient method to generate integration-free human iPS cells” Nature Methods, Vol. 8 No. 5, May 2011, pages 409-411 or by Lu et al “A defined xeno-free and feeder-free culture system for the derivation, expansion and direct differentiation of transgene-free patient-specific induced pluripotent stem cells”, Biomaterials 35 (2014) 2816e2826.

As explained herein, the (induced) pluripotent stem cell that is used in the present invention can be derived from any suitable cell type (for example, from a stem cell such as a mesenchymal stem cell, or an epithelial stem cell or a differentiated cells such as fibroblasts) and from any suitable source (bodily fluid or tissue). Examples of such sources (body fluids or tissue) include cord blood, skin, gingiva, urine, blood, bone marrow, any compartment of the umbilical cord (for example, the amniotic membrane of umbilical cord or Wharton's jelly), the cord-placenta junction, placenta or adipose tissue, to name only a few. In one illustrative example, is the isolation of CD34-positive cells from umbilical cord blood for example by magnetic cell sorting using antibodies specifically directed against CD34 followed by reprogramming as described in Chou et al. (2011), Cell Research, 21:518-529. Baghbaderani et al. (2015), Stem Cell Reports, 5 (4): 647-659 show that the process of iPSC generation can be in compliance with the regulations of good manufacturing practice to generate cell line ND50039.

Accordingly, the pluripotent stem cell preferably fulfils the requirements of the good manufacturing practice.

In the method of the present invention, the culture medium in the closed bioreactor system is advantageously continuously replaced by fresh medium. This can be done by a strategy named “vessel settling strategy”, by a strategy named “tip settling” or by perfusion, e.g., by applying a rotating mesh such as a spin filter. In the “vessel settling” strategy, which can be seen as a batch method, the agitation in a STR is stopped, allowing the aggregates to sediment to the bottom of the vessel. Subsequently, medium is removed without disturbing the aggregates, fresh medium is added and the agitation is started again. The second and preferred strategy, the “tip settling” strategy is described in WO 2021/116361. It may be used to simulate perfusion medium exchange. Here, a small amount of medium is aspirated by a pipet comprised by the closed bioreactor system for a defined duration. During this time the aggregates sediment at the tip of the pipet tip. Finally, a part of the aspirated medium, including the settled aggregates, is returned to the vessel, whereas the rest is discarded. Afterwards, fresh medium is added. The remaining aggregates in the vessel are continuously stirred during the entire procedure. The “tip settling” strategy described herein is especially preferred for step (0) of the method of the invention. The “vessel settling” strategy described herein (c.f. also the description regarding the “washing” of the cells during medium change) is especially preferred for medium changes between steps (i) to (iv) of the method of the invention.

The culture medium may be continuously exchanged using perfusion in the method of the invention. Perfusion is characterized by the continuous replacement of medium from the reactor by fresh medium while retaining cells in the vessel by specific systems. Perfusion is an operation mode for biopharmaceutical production processes enabling highest cell densities and productivity. Beside the advantage that cells in perfusion are constantly provided with fresh nutrients and growth factors, potentially toxic waste products are washed out, ensuring more homogeneous conditions in the reactor. Moreover, compared to repeated batch processes, perfusion processes support process automation and improved feedback control of the culture environment, including DO, pH, and nutrient concentrations. Perfusion cultures may enable a relatively stable, physiological environment that also supports the self-conditioning ability of PSCs by their endogenous factor secretion and thus eventually reducing supplementation of expensive medium components.

When applying a rotating mesh such as a spin filter, which is also preferred, medium is continuously exchanged by perfusion through the spin filter while the cells remain in the closed bioreactor system. Spin filters are especially preferred for medium change in step (i), step (ii), step (ii) (a), step (iii) and step (iv) of the method of the invention. Accordingly, step (i), step (ii), step (ii) (a), step (iii) and step (iv) of the method of the invention can be conducted comprising perfusion of the medium, preferably by applying a spin filter to the closed bioreactor system. Additionally, also the optional step (0) of the method of the invention can be conducted comprising perfusion of the medium, preferably by applying a spin filter to the closed bioreactor system.

The amount of medium replaced by fresh medium can be described as perfusion per day in every strategy for medium exchange. Typical exchange rates include 40% to 60%, preferably about 50% of the medium comprised in the closed bioreactor system by perfusion per day. In step (0) of the method of the invention, the perfusion rate preferably is 50% to 70%, more preferably about 60% per day. In step (i) of the method of the invention, the perfusion rate is preferably about 100% per day.

Between the different steps of the method of the invention, the medium preferably is exchanged. The medium change may be complete or partial. Accordingly, in the method of the present invention, a change of the medium preferably is conducted at least at the transition from step (0) to step (i), from step (i) to step (ii), from step (ii) to step (ii) (a), from step (ii) (a) to step (iii), and from step (iii) to step (iv). The medium change at the transition from step (i) to step (ii), from step (ii) to step (ii) (a), from step (ii) (a) to step (iii), and from step (iii) to step (iv) preferably is complete. The medium change at the transition from step (0) to step (i) preferably is partial.

Each medium change preferably includes a step of washing the cells comprised in the closed bioreactor system. An exemplary washing method is described in the following: To switch medium types the agitation of the STR is stopped for a time sufficient for sedimentation of the cell aggregates. The medium can then be aspirated with a dip tube that is set to a certain height so that the settled aggregates are not discarded. The following procedure can be performed to switch between medium types:
1. Stop agitation
2. Aggregate sedimentation (e.g., for about 5 min; depends on vessel height)
3. Aspiration of medium with dip tube of fixed height
4. Addition of new medium type (5-times the volume of the remaining medium in the vessel)
5. Agitate, e.g., for 10 s to ensure even distribution
6. Stop agitation
7. Aggregate sedimentation (~5 min; depends on vessel height)
8. Aspiration of medium with dip tube of fixed height
9. Addition of new medium type (5-times the volume of the remaining medium in the vessel)
10. Agitate, e.g., for 10 s to ensure even distribution
11. Stop agitation
12. Aggregate sedimentation (e.g., for about 5 min; depends on vessel height)
13. Aspiration of medium with dip tube of fixed height
14. Addition of new medium type to culture volume
15. Start agitation (again).

Alternatively, the washing steps (1-10) may be performed with the basal medium instead of the complete new medium to save expensive medium factors. In this case, the volume that remains in the vessel has to be known and concentrated complete medium has to be added in step 14 to make the final working concentration after addition to the remaining medium in the vessel.

In order to enable a smooth medium transition from the PSC expansion step (0) that may be part of the method of the invention to the culture medium applied in step (i) of the method of the invention, the medium change from the culture conditions of step (0) to that of step (i) can be partial. A partial medium change in this context means that up to 75%, preferably up to 50%, preferably between 5 and 75%, more preferably between 5 and 50%, more preferably between 5 and 40%, most preferably about 25% v/v of the medium used in step (0) are comprised in the medium of step (i). Accordingly, the change of medium from step (0) to step (i) may comprise a partial medium change, preferably wherein the medium of step (0) remains unchanged for an amount of about 5 to 75% v/v, preferably 25% v/v.

The term “suspension culture” as used herein is a type of cell culture in which single cells or small aggregates of cells are allowed to function and multiply in an agitated growth medium, thus forming a suspension (c.f. the definition in chemistry: “small solid particles suspended in a liquid”). This is in contrast to adherent culture, in which the cells are attached to a cell culture container, which may be coated with proteins of the extracellular matrix (ECM). In suspension culture, preferably no proteins of the ECM are added to the cells and/or the culture medium.

As used herein, the terms “reactor” and “,bioreactor”, which can be used interchangeably, refer to a closed culture vessel configured to provide a dynamic fluid environment for cell cultivation. Examples of agitated reactors include, but are not limited to, stirred tank bioreactors, wave-mixed/rocking bioreactors, up and down agitation bioreactors (i.e., agitation reactor comprising piston action), spinner flasks, shaker flasks, shaken bioreactors, paddle mixers, vertical wheel bioreactors. An agitated reactor may be configured to house a cell culture volume of between about 2 mL-20,000 L. Preferred bioreactors may have a volume of up to 50 L. An exemplary bioreactor suitable for the method of the present invention is the ambr15 bioreactor available from Sartorius Stedim Biotech. Further suitable bioreactors include the UniVessel system also available from Sartorius Stedim Biotech. Further suitable bioreactors are commercially available, e.g., from General Electric or Eppendorf. The pH of the culture medium may be controlled by the bioreactor, preferably controlled by CO₂ supply, and may be held in a range of 6.6 to 7.6, preferably at about 7.4.

A “closed bioreactor system” as used herein relates to a bioreactor configuration, which allows carrying out the method of the invention without removing the cells prior to the final harvest from the bioreactor. In other words, the method of the invention preferably does not include a step of removing cells during any one of steps (0) to (iv). Additionally, the method of the invention preferably does not include a step of adding cells during any one of steps (i) to (iv), preferably the method of the invention preferably does not include a step of adding cells during any one of steps (0) to (iv). Removal of cells or addition of further cells can be avoided by the techniques described herein.

In some embodiments, the volume of the culture vessel in the bioreactor is from about 50 mL to about 20,000 L. In some embodiments, the volume of the culture vessel in the bioreactor is from about 50 mL to about 2,000 L. In some embodiments, the volume of the culture vessel in the bioreactor is from about 50 mL to about 200 L. In some embodiments, the volume of the culture vessel in the bioreactor is from about 50 mL to about 100 L. In some embodiments, the volume of the culture vessel in the bioreactor is from about 50 mL to about 50 L. In some embodiments, the volume of the culture vessel in the bioreactor is from about 50 mL to about 20 L. In some embodiments, the volume of the culture vessel in the bioreactor is from about 50 mL to about 10 L. In some embodiments, the volume of the culture vessel in the bioreactor is from about 50 mL to about 1 L. In some embodiments, the volume of the culture vessel in the bioreactor is from about 100 mL to about 10 L. In some embodiments, the volume of the culture vessel in the bioreactor is from about 100 mL to about 5 L. In some embodiments, the volume of the culture vessel in the bioreactor is from about 150 mL to about 1 L. In some embodiments, the volume of the culture vessel in the bioreactor is from about 1 L to about 1,000 L.

Especially preferred are bioreactors, in which the minimal and maximal cell culture volume differs by 5-fold or even 10-fold, i.e. bioreactors that can be understood to allow upscaling in the same bioreactor. Such a bioreactor may allow the start of PSC expansion in a relatively small volume, such as 200 mL. If the cell dissociation reagent is diluted by an excess volume of the culture medium, e.g. a 5-fold addition of cell culture medium, this yields a final volume of about 1 L after the first passage. After cell expansion, the cells are then separated again and the subsequent addition of an excess volume of culture medium then increases the volume to, e.g., 5 L after the second passage of the cells. Thus, in bioreactors that accept both relatively small and large volumes, the cells can be passaged in the same bioreactor several times without any manual operation (in a cascade-like process), e.g. removing a part of the cells and using this part to inoculate a further bioreactor while the remaining fraction of the cells is used to inoculate the bioreactor again (“repeated batch strategy” or “cascade-like process”).

This allows the expansion of PSCs by around 1000-fold without any manual interaction such as transfer of cells in and out of the bioreactor necessary.

The method of the invention may be suitable for use at large-scale (e.g., between 1 I to 1000 I). In one preferred embodiment, for large scale production, the bioreactor suitable for use in the second or subsequent culture period(s) is a larger reactor than the bioreactor used for initial culture and dissociation. In one preferred embodiment, multiple bioreactors are inoculated in parallel for use in the second or subsequent culture period(s), thereby facilitating parallel serial passaging.

The bioreactor may be an agitated bioreactor or a stirring bioreactor. The speed of the stirrer preferably is optimized for each individual bioreactor. A person skilled in the art is capable of selecting a speed of the stirrer suitable for culturing of PSCs and dissociation of PSC cell aggregates. The speed of the stirrer for culturing of the PSCs preferably is lower such as in the range of 150 to 450 rpm, preferably about 300 rpm, in contrast to the speed suitable to facilitate cell dissociation, which might require a higher speed such as in the range of 450 rpm to 750 rpm, preferably about 600 rpm. For washing, the stirring speed preferably is in the range of 150 to 450 rpm, preferably about 300 rpm. Accordingly, in one embodiment the bioreactor is the ambr15 bioreactor of Sartorius Stedim and the stirring speed is 300 rpm for cell growth and 600 rpm for cell dissociation.

Another condition that determines whether the conditions are suitable, e.g., for the expansion of the PSC, for mesodermal induction, for inducing differentiation and/or conducting metabolic selection, includes temperature. Accordingly, wherein the temperature of the culture medium is about 30 to 50° C., about 35 to 40° C., about 36 to 38° C. or about 37° C., preferably 37° C.

The present invention further relates to a population of differentiated cells obtainable by the method of the invention.

The present invention further relates to a population of differentiated cells obtained by the method of the invention.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

As used herein the term “about” or “approximately” means within 20%, preferably within 15%, preferably within 10%, and more preferably within 5% of a given value or range. It also includes the concrete number, i.e. “about 20” includes the number of 20.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXPERIMENTAL EXAMPLES

An even better understanding of the present invention and of its advantages will be evident from the following experimental examples, offered for illustrative purposes only. The examples are not intended to limit the scope of the present invention in any way.

### Materials

### Cell Line

TC1133: TC1133 is a human iPS cell line that was generated by Lonza under cGMP-compliant conditions (Baghbaderani et al., 2015, 2016).

### Equipment

Mini-bioreactor system: ambr15 cell culture with cooling; Sartorius Stedim Biotech;
pH-Meter: Multi 3510 IDS; Xylem Analytics Germany GmbH
pH-Electrode: SenTix Micro 900P; WTW
Cell counter: Nucleocounter NC-200 Type 900-0201, Chemometec
Automated cell imager: Cellavista V 3.1; SynenTec
Flow Cytometer
LSR II Special Order System; BD
CytoFlex; Beckman Coulter
Bioreactor: Biostat B-DCU II; Sartorius Stedim Biotech; Type: BB-8841212; Tower 3: Type: BB-8840152, pH Sensor for UniVessel 0.5L: Hamilton; Easyferm Plus VP; Oxygen Sensor for UniVessel 0.5L: Hamilton; Oxyferm FDA VP 120; Tower 2: Type: BB-8840152; pH Sensor for UniVessel 0.5L: Hamilton; Easyferm Plus VP; Oxygen Sensor for UniVessel 0.5L: Hamilton; Oxyferm FDA VP 120; pH Sensor for UniVessel 2L: Hamilton; Easyferm Plus PHI VP 225 Oxygen Sensor for UniVessel 2L: Hamilton; OxyFerm FDA VP 225.
Futura Tool: Part #: 234300
Viamass: Part #: 6530-52-03
UniVessel 0.5L equipped with spinfilter
UniVessel 2L equipped with spinfilter

### Consumables

ambr15 cell culture 24 Disposable bioreactors, Low temp, no sparge; Sartorius Stedim Biotech; Part number: 001-2B81

### Cell Culture Media and Reagents

Cell culture media and reagents that are established in adherent culture of iPSC and iPSCCM were used as a basis for suspension culture:
StemMACS iPS-Brew XF, Basal Medium; Miltenyi Biotec; Cat. no.: 130-107-086
StemMACS iPS-Brew XF 50× Supplement; Miltenyi Biotec; Cat. no.: 130-107-087
RPMI 1640 Medium; Thermo Fisher Scientific; Cat. no.: 11875093
ROCKi Y27632; Stemolecule; Cat. no.: 04-0012-10
Versene Solution, Thermo Fisher Scientific; Cat. no.: 15040-033
CHIR99021; Stemgent; Cat. no.: 04-0004-10
IWP4; Peprotech; Cat. no.: AF-120-05ET
Recombinant Human/Mouse/Rat Activin A Protein; Bio-Techne; 338-AC
Recombinant Human BMP-4; R&D Systems, 314-BP
Animal-Free Recombinant Human FGF-basic (154 a.a.); Peprotech; AF-100-18B
B-27 Supplement (50X), serum free; Thermo Fisher Scientific; Cat. no.: 17504044
B-27 Supplement, minus insulin; Thermo Fisher Scientific; Cat. no.: A1895601
Laminin; Biolamina; Biolaminin 521 MX (MX521)
TrypLE Express Enzyme (1X), no phenol red; Thermo Fisher Scientific; Cat. no.: 12604013
DPBS, no calcium, no magnesium; Thermo Fisher Scientific; Cat. no.: 14190094
Roti-Histofix 4%, acid free (pH 7), phosphate-buffered formaldehyde solution 4%; Carl Roth, Cat. no.: P087.6
DMSO; Sigma; Cat. no.: D2650-100ML

### Methods

### Suspension Culture

To establish the suspension culture of iPSCs in this project, the ambr15 system was chosen. It allows for an efficient screening of culture conditions during the proof of concept stage and the optimization stage. This is because the ambr15 system controls up to 24 mini-bioreactors in parallel with different culture conditions. At the same time, the system is cost efficient because the maximal working volume of medium per minibioreactor is only 15 mL. Using the ambr15 system, the culture temperature, pH, d0, stirring speed and stirring direction may be monitored and controlled. Therefore, the system holds promise for a successful transfer of culture strategies to a larger bioreactor system.

The Biostat B-DCU II system in combination with the UniVessel 0.5L system was used to show proof of concept for the upscaling of the developed expansion strategy.

### Analysis of Expansion Rate

The nucleocounter NC-200 offers fast and reliable assessment of cell number and viability. Furthermore, it is GMP-compliant and is used during the production stage. Single cell suspensions may be directly measured with the NC-200, whereas suspensions with high content of cell clumps should be treated according to special protocols, supplied with the NC-200. These protocols include cell lysis to ensure distinguishability of single nuclei. However, large iPSC aggregates of about 70-250 μm, which were obtained in the suspension culture, could not be sufficiently lysed with the provided protocols within a reasonable time. Therefore, iPSC aggregates were digested using TrypLE before measurement with the NC-200. That way, a near single cell suspension was achieved, which was comparable to detached iPSCs during monolayer culture. This suspension was then used for measurement with the NC-200 using the “Viability and Cell Count—A100 and B Assay” intended for small cell aggregates.

### Analysis of Gene Expression

The pluripotency state of iPSCs was assessed by analyzing the pluripotency-related markers NANOG, TRA-1-60, and OCT4 using flow cytometry. iPSC aggregates were digested with TrypLE to achieve a near singe cell suspension. Subsequently the cells were fixed by adding Histofix to the cell suspension in a 1:1 ratio (resulting in 2% PFA). For analysis, the cells were stained with fluorochrome-conjugated antibodies. Dead cells were excluded from the analysis with Hoechst staining. Accordingly, the expression of a-Actinin and cTNT was analyzed to assess the success of cardiac differentiation.

### Analysis of Aggregate Size and Morphology

The quality of iPSC aggregate morphology was assessed visually by light microscopy. A good aggregate morphology is characterized by a round shape and smooth borders. However, the aggregates should not be perfectly round, but dent-like structures should be visible. Furthermore, the iPSCs in an aggregate should appear homogeneous and no vacuoles or dense regions should be observable.

The aggregate size was analyzed using a Cellavista device. Culture samples were transferred into a 24-well plate prepared with PBS (—Ca2+/—Mg2+) and measured with the Cellavista. Images were processed by the Cellavista software by applying a threshold to discriminate aggregates from background. The software FIJI (Schindelin et al., 2012) was subsequently used to analyze aggregate size.

### Cardiac Differentiation

### Ambr15

The cardiac differentiation potential of iPSCs after long-term culture was assessed by direct differentiation into iPSC-CMs using a published 2-factor protocol (Chen et al., 2015). iPSC aggregates were differentiated in the ambr15 system and the medium was exchanged manually. Briefly, the basal medium on day 0-5 consisted of RPMI 1640+2% B-27 supplement without insulin. From day 6 onwards the basal medium was RPMI 1640+2% b-27 supplement. To initiate mesodermal differentiation, CHIR was added in varying concentrations on day 1. 5 μM IWP4 was added on day 2 and 3 or day 3 and 4 to initiate cardiac differentiation.

### UniVessel

iPSC aggregates that were generated in the UniVessel were directly differentiated into cardiomyocytes using the ABCF differentiation protocol. Briefly, Activin-A (9 ng/ml), BMP-4 (5 ng/ml), CHIR (2 ng/ml) and bFGF (5 ng/ml) were applied during day 0-2 of differentiation. On day 3-9, the cells were treated with 5 μM IWP4. The differentiation was performed in basal serum-free medium (BSFM; RPMI-1640+1 mM sodium pyruvate+2% B27-Supplement+200 μM ascorbic acid). On day 0, different ratios of BSFM and iPSbrew complete were tested. On day 0-3 B27-Supplement without insulin was used. Metabolic selection was performed on day 12-19 by exchanging the BSFM for selection medium (RPMI-1640 without glucose+2.82 mM lactate+100 μM 2-Mercaptoethanol). The differentiation was completed on day 23 and cells were harvested. To singularize aggregates, the cells were digested using Collagenase I or II and TrypLE 1× or 10×.

### Example 1: Aggregate Formation

To establish conditions for inoculation that favor the formation of iPSC aggregates in STRs, a proof of concept run was performed with 24 ambr15 mini-bioreactor vessels (Proof of concept run 01). Culture conditions were initially chosen based on experiences with adherent culture of iPSCs. Accordingly, culture temperature of 37° C. and a pH of 7.4 were chosen.

According to the literature, the stirring speed can influence iPSC aggregate formation. This is because extensive stirring causes shear stress to the cells. Therefore, low stirring speeds of 300 rpm and 600 rpm in a downward direction were chosen for testing. 300 rpm is the lowest stirring speed possible for the standard setup of the ambr15.

Furthermore, the seeding density is described to influence the formation of iPSC aggregates. Based on the literature, seeding densities ranging from 5×10⁴ to 5×10⁵ cells/mL were chosen. Additionally, the influence of oxygen concentration on aggregate formation was assessed by testing 10%, 50% and 90% oxygen (air saturation).

To inoculate the ambr15 mini-bioreactors, iPSCs grown in adherent cell culture on Laminin were used. The cells were detached non-enzymatically with Versene, resulting in a cell suspension containing clumps of ~10 cells. iPSC aggregates were observed in the suspension culture on the first day after inoculation at all tested conditions. Using Design of Experiments (MODDE), the factors seeding density and stirring speed were found to be crucial for both the aggregate size and cell number one day after inoculation. The increase of seeding density causes both larger aggregates and higher cell number on day one. Stirring at 600 rpm caused smaller aggregate size and cell number than stirring at 300 rpm. The aggregate size (~100 μm) and morphology on day 1 at 300 rpm was in the optimal range of 100-400 μm described in the literature.

Based on the results of Proof of concept run 01, the culture parameters temperature (37° C.), pH 7.4, stirring speed (300 rpm) and stirring direction (downward) were fixed for further experiments. The seeding density, oxygen concentration and culture volume were further optimized.

### Example 2: Medium Exchange

IPSC aggregates were successfully generated in Proof of concept run 01. However, the prolonged cultivation failed during this run because of an insufficient strategy for the exchange of medium. Therefore, two optimized medium exchange strategies were tested in Medium Change Optimization Run 01. Both strategies were meant to enable the exchange of culture medium during an ambr15 run at maximum capacity of 24 vessels while maintaining pluripotency and vitality of the iPSCs. Also, the Inventors aimed for a minimal loss of cells during medium exchange.

The first strategy was named “vessel settling strategy” and was based on reports of larger STR. Here, the agitation is stopped, allowing the aggregates to sediment to the bottom of the vessel. Subsequently, medium is removed without disturbing the aggregates, fresh medium is added and the agitation is started again. The medium exchange of the remaining 23 vessels was simulated by stopping and starting the agitation for respective time periods. 66% medium was exchanged at each cycle.

The second strategy was termed “tip settling strategy”, which is also described in WO 2021/116361. It may be used to simulate perfusion medium exchange. Here, a small amount of medium is aspirated by the ambr15 and remains in the pipet for a defined duration. During this time the aggregates sediment at the tip of the pipet. Finally, a part of the aspirated medium, including the settled aggregates, is returned to the vessel, whereas the rest is discarded. Afterwards, fresh medium is added. The aggregates in the vessel are continuously stirred during the entire procedure. The cycle of removing and adding medium was repeated until 57% medium was exchanged overall.

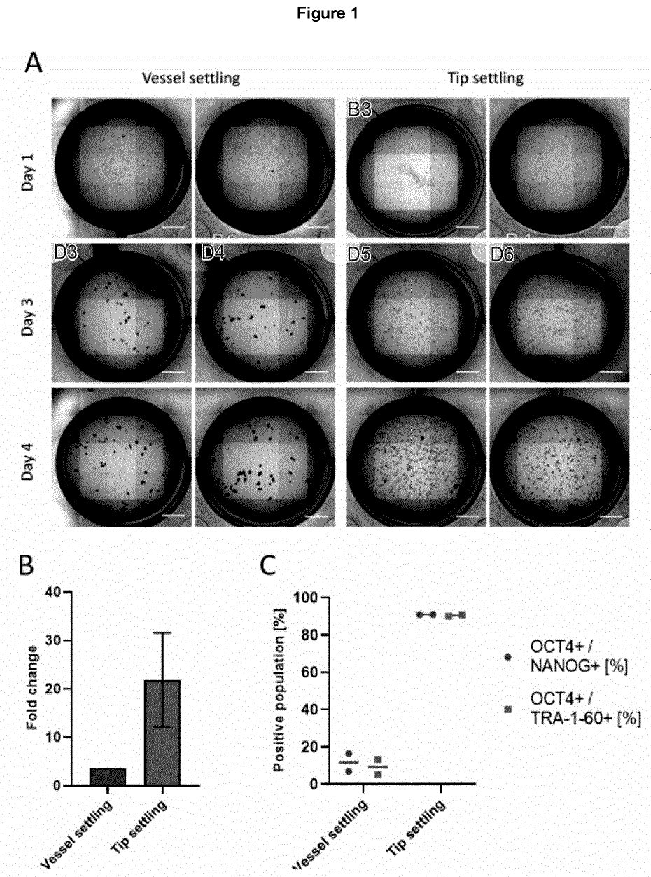

Significant differences were observed when comparing the outcome of both medium exchange strategies (FIG. 1). After four days of culture, the aggregates of the tip settling strategy had a good morphology with homogeneous aggregate sizes, the mean expansion rate was about 20-fold and the expression of pluripotency-related genes was over 90% (OCT4/NANOG and OCT4/TRA-1-60 double positive population). On the other hand, the expansion rate in aggregates of the vessel settling strategy was about 4-fold and the expression of pluripotency-related genes was below 20%. Furthermore, fusion of aggregates was observed in the vessel settling culture, causing a heterogeneous, polymorphic morphology of aggregates. The poor quality of iPSCs in the vessel settling culture was most likely a result of the accumulation of time without stirring. During this time, the iPSC aggregates would fuse and spontaneously differentiate.

As a result of the Medium Change Optimization Run 01, the vessel settling strategy was dismissed for the medium exchange of an iPSC suspension culture in a maximum capacity run in the ambr15. However, the strategy may still be useful for a single vessel, where the time without agitation can be kept to a minimum. Subsequently to Medium Change Optimization Run 01, the tip settling strategy was optimized and following parameters were established for the following experiments: 1000 μL medium uptake, 5 min pause, 100 μL medium return to vessel, 900 μL medium discarded.

### Example 3: Optimization of Culture Conditions

A series of experiments were performed, termed Cultivation Optimization Runs, to optimize the culture parameters culture volume, seeding density, beginning of medium exchange, volume of medium exchange per day and oxygen concentration.

It was found that a small cultivation volume of 10 mL causes decreased aggregate size and expansion rate (Cultivation Optimization Run 03). On the other hand, a large cultivation volume (e.g. 15 mL) decreases the potential maximal amount of medium that can be exchanged per day. Therefore, the cultivation volume of 13 mL was found to be optimal for the suspension culture of iPSCs in the ambr15.

It was found that the seeding density at day 0 (transfer from adherent to suspension culture of iPSCs) influences the aggregate size and expansion rate. On the one hand, larger aggregates form at higher seeding densities (e.g. 5×10⁵ cells/mL) compared to small densities (e.g. 1×10⁵ cells). On the other hand, a trend that the expansion rates were higher at smaller seeding densities was observed. Furthermore, results from Cultivation Optimization Run 03 indicated a reduced expression of pluripotency-related genes at high seeding densities. Overall, the seeding density of 2.5×10⁵ cells/mL at day 0 was found to result in robust formation of aggregates with decent size at day 1 and 4 after inoculation (~100 μm and ~200 μm, respectively), high expansion rates (~10 fold after four days in passage 0) and high expression of pluripotency-related genes (>95%).

Cultivation Optimization Run 11 focused on the optimization of volume of exchanged medium per day and starting time point of medium exchange. Strong evidence was found that the beginning of medium exchange at day 2 results in a higher expansion rate than beginning at day 1 after inoculation (14.6-fold against 12.8-fold). At the same time, the expression of pluripotency-related genes remained high. The exchange of 62% medium per day (meaning 9 cycles of tip settling medium exchange) resulted in high quality iPSC aggregates after four days. On the other hand, an exchange of 90% medium resulted in a mild decrease of pluripotency-related genes (<40% NANOG positive cells). However, this effect may also be artificially caused by the ambr15 tip settling strategy, since an increase in cycles causes an increased chance of aggregate fusion. Taken together, the exchange of 62% medium beginning at day two after inoculation was established for the suspension culture of iPSCs in the ambr15.

The oxygen concentration in cell media of ~18% in regular cell culture using CO2-incubators is highly artificial compared to the in vivo oxygen concentration ranging from 4-7% depending on the organ (Ast and Mootha, 2019). Furthermore, it has been reported that cultivation at normoxia or hypoxia results in an increased yield of PSCs and PSC-CMs (Correia et al., 2014; Forsyth et al., 2006; Niebruegge et al., 2009). Therefore, we analyzed the influence of oxygen concentration on the iPSC suspension culture in the ambr15. Cultivation Optimization

Runs 05 and 07 showed a mild increase in iPSC quality at passage 0 resulting from reduced oxygen concentration. Interestingly, reduced oxygen concentration was found to cause faster aggregate dissociation during passaging and a higher quality of iPSCs during passage 1 (Cultivation Optimization Run 07). For these reasons, the oxygen concentration of 28.3% (air saturation) was established for the suspension culture of iPSCs in the ambr15, which is equivalent to normoxia (5% oxygen concentration in the medium).

Taken together, the following cultivation parameters were established:

TABLE 4
Optimized culture conditions for the suspension
culture of iPSCs in the ambr15 system.

| Parameter | Detail | |
|---|---|---|
| Temperature | 37° | C. |
| pH | 7.4 | |
| Oxygen concentration | 23.8% air saturation | |
| Stirring speed | 300 | rpm |
| Stirring direction | Downwards | |
| Cultivation volume | 13 | mL |
| Seeding density | 2.5 × 10⁵ | cells/mL |
| Medium exchange volume | 62%/day | |
| Beginning of medium exchange | Day | 2 |

### Example 4: Passaging

The development of a strategy for passaging was divided into two goals. First, conditions were screened that result in dissociation of aggregates into a homogeneous cell suspension with high viability. Subsequently, a strategy for reassembly of iPSC aggregates had to be established. Aggregates that were generated in the Cultivation Optimization Runs were used to develop a procedure for passaging.

Dissociation of aggregates was tested using Versene, Accutase and TrypLE, all of which are either used for detachment of iPSCs during passaging of adherent cultures or have already been described for iPSC aggregate dissociation. In the beginning, dissociation was tested both manually and in the ambr15. Later, automatic dissociation in the ambr15 was optimized. Therefore, the stirring speed was increased during dissociation to supply additional mechanical force. Dissociating the aggregates at 600 rpm for up to 15 min was found to be suitable. Since iPSCs become apoptotic when singularized, dissociation of aggregates into clumps of about 1-50 cells was aimed for. Based on the procedures to detach iPSCs in adherent cell culture, aggregates were washed either with the dissociation reagent or with PBS. All tested dissociation reagents dissociated the aggregates. However, the viability, single-cell state and homogeneity varied between experiments. Overall, Versene and Accutase were found to be suitable for aggregate dissociation, whereas the use of TrypLE often caused a high degree of cell singularization.

To develop a method for reaggregation of iPSCs, the dissociated cells were centrifuged in the initial experiments to remove the dissociation reagents. However, the goal was to develop a process that does not include centrifugation steps. For this reason, it was tested to directly transfer the cells in the dissociation reagent into the fresh culture medium. Newly formed aggregates were observed with all dissociation reagents either with or without removal of the reagent during inoculation. However, the quality of passage 1 aggregates was often not sufficient. Furthermore, the variance was high and in some experiments very few aggregates were observed in passage 1.

To increase the degree of reaggregation, pretreatment with ROCK inhibitor Y27632 was tested. Pretreatment of iPSCs with Y27632 has been reported for other procedures that require cell singularization and are stressful for iPSCs (Chatterjee et al., 2011). In Cultivation Optimization Run 08, the passaging with Versene and Accutase was compared with and without Y27632 pretreatment. The results show that dissociation with Versene is more suitable than Accutase for the passaging of iPSCs in the ambr15. Only few aggregates of poor quality were observed in passage 1 when using Accutase, regardless of the Y27632 pretreatment. On the other hand, automatic passaging in the ambr15 with Versene, resulted in the formation of iPSC aggregates in passage 1 with decent quality.

Interestingly, the pretreatment with Y27632 resulted in higher expansion rates on day 5 (pretreatment: 9-fold; untreated: 5-fold), larger aggregates (pretreatment: 162 μm mean; untreated: 114 μm mean) and higher expression of NANOG on day 3 (pretreatment: 93%; untreated: 62%) in passage 1.

Ultimately, the following procedure for the automatic passaging of iPSCs in the ambr15 was tested in the Cultivation Optimization Run 11:
Y27632 treatment of iPSC aggregates at a final concentration of 10 μM two hours before dissociation.
Two times washing with Versene: Stop of stirring for two minutes, removal of medium to 2 mL without disturbing sedimented aggregates, addition of Versene to 10 mL, starting the stirring (300 rpm, downwards) for 10 seconds.
Removal of medium to 2 mL in the same way as described in the washing steps and addition of Versene to 5 mL.
Stirring at 600 rpm for up to 15 min until dissociation is sufficient. In-Process controls have to be observed microscopically to assess the right degree of dissociation.
Reduce stirring to 300 rpm.
Cell count.
Transfer a volume of cell suspension into a fresh ambr15 vessel resulting in a seeding concentration of 5×10⁵ cells/mL.

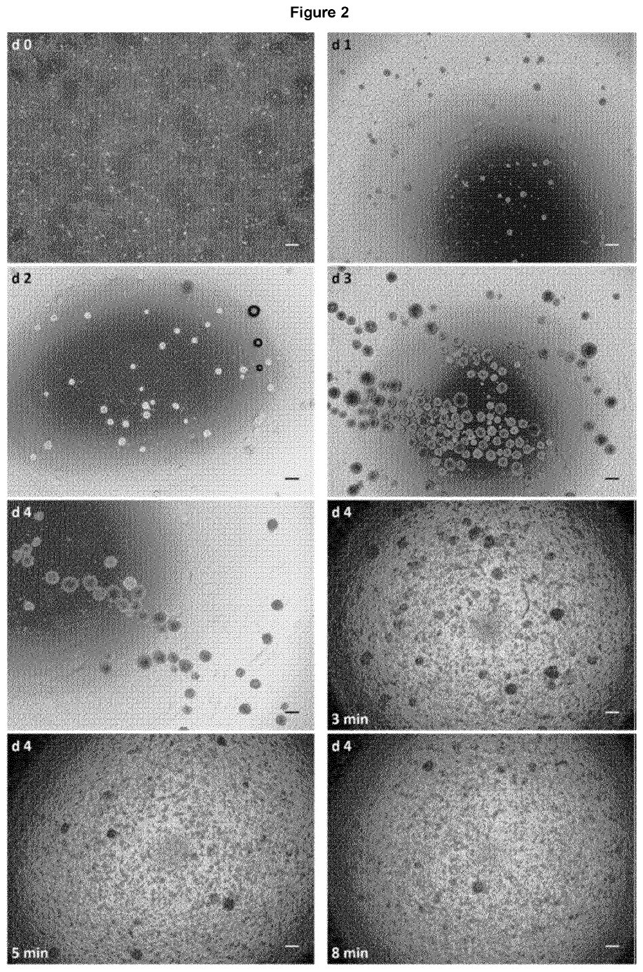

iPSC aggregates with high quality were generated (FIG. 2). During this run, three consecutive passages were performed. At passage 3, the accumulated expansion rate was 1043-fold and a high expression of pluripotency-related genes was found (>90% double positive population). Furthermore, it was shown that iPSCs still form aggregates up to a 1:5 dilution of transferred Versene. This passaging procedure was used for later experiments.

### Example 5: Long Term Culture

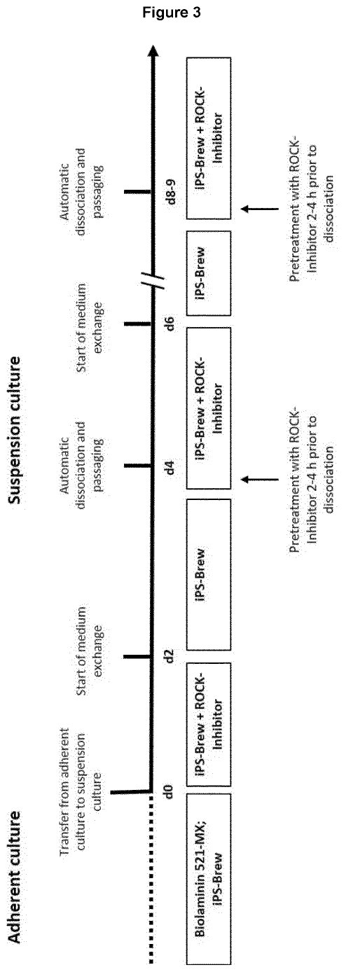

The established strategies for aggregate formation (Example 1), cell expansion (Examples 2 and 3) and passaging (Example 4) were used in Cultivation Optimization Run 12 and 13 to test a sustained suspension culture of iPSCs (FIG. 3). iPSCs were passaged ten times in Cultivation Optimization Run 12 and eight times in Cultivation Optimization Run 13. The cells were cultured for four to five days in each passage (except for passage 6 and 8 of Cultivation Optimization Run 12, which lasted three days).

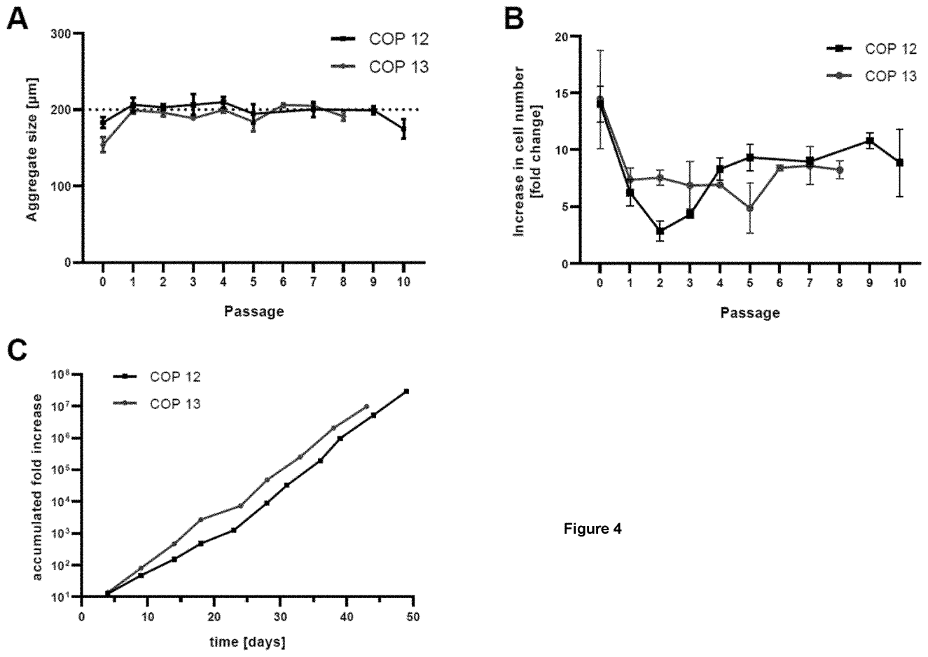

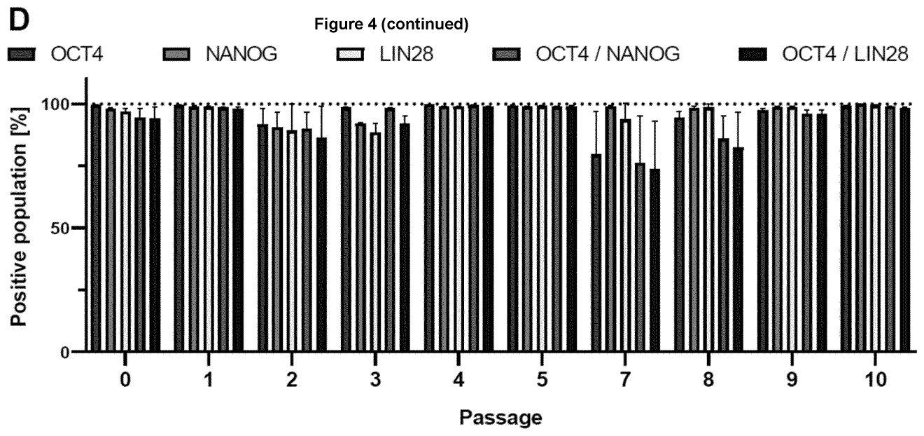

The iPSC aggregates depicted a good morphology in all passages. The aggregate size was about 200 μm at the end of each passage that lasted 4-5 days (FIG. 4A).

Interestingly, the expansion rate was highest at passage 0 with about 14-fold increase in cell number (FIG. 4B). At later passages (about 5-10) the expansion rate was about 8-fold in each passage that lasted 4-5 days. The expansion rate was low (about 4-fold) in passages 1-3 of Cultivation Optimization Run 12. However, passages 1-5 of Cultivation Optimization Run 13 had an expansion rate of about 7-fold. The calculated accumulated expansion rate over the complete cultivation time shows an exponential growth of iPSCs (FIG. 4C). An accumulated fold increase of 2.9×10⁷ after 49 days and 9.6×10⁶ after 43 days was reached in Cultivation Optimization Run 12 and 13, respectively.

A high expression of pluripotency-related genes was found at the end of individual passages throughout the entire long-term culture (FIG. 4D). Following intervals for passaging were found to generate iPSCs of high quality:

TABLE 5
Exemplary passaging strategy for iPSC suspension
culture in the ambr15 system.

| | | | Exemplary weekday |
|---|---|---|---|
| Days of | | Days in | of passage start |
| culture | Passage | passage | (d 0) |
| 0-4 | 0 | 4 | Monday |
| 5-9 | 1 | 5 | Friday |
| 10-14 | 2 | 5 | Wednesday |
| 15-18 | 3 | 4 | Monday |
| 19-24 | 4 | 5 | Friday |
| 25-29 | 5 | 5 | Wednesday |
| 30-33 | 6 | 4 | Monday |
| 34-38 | 7 | 5 | Friday |
| 39-43 | 8 | 5 | Wednesday |
| 44-47 | 9 | 4 | Monday |
| 48-52 | 10 | 5 | Friday |

### Example 6: Cardiac Differentiation of iPSCs in Suspension

A main goal of this project was to ensure that the cardiac differentiation potential remains in iPSCs that are cultivated with the developed expansion strategy. The cardiac differentiation potential was assessed by using a published protocol for the directed cardiac differentiation of PSCs in suspension culture (Chen et al., 2015).

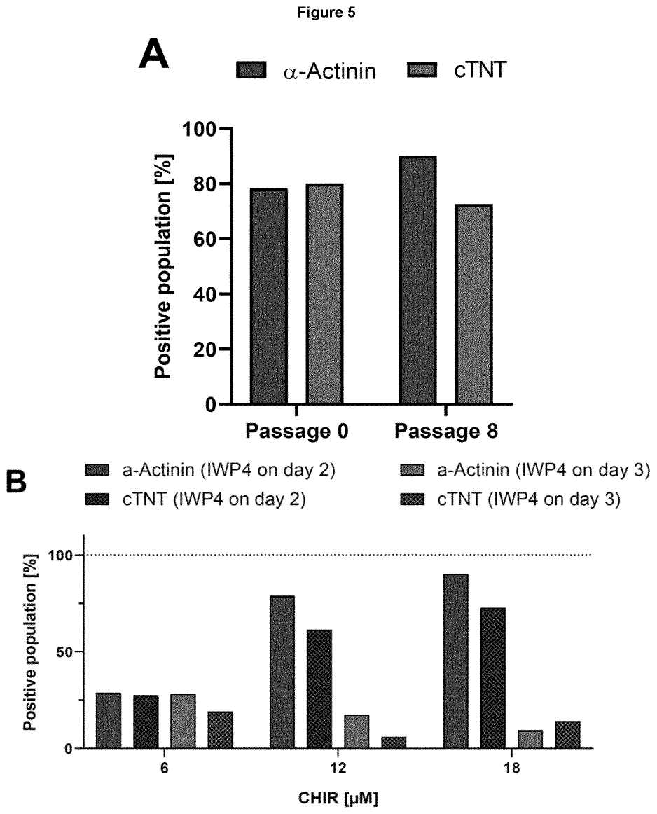

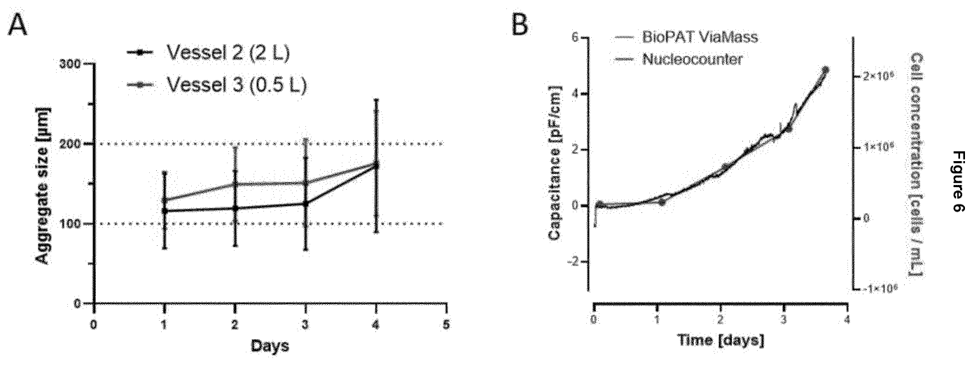

iPSCs in suspension were successfully differentiated into iPSC-CMs during early passages (Cultivation Optimization Run 12) and during late passages (Cultivation Optimization Run 13) of long-term cultures. Thereby, proof of concept was shown that iPSCs generated with the developed expansion strategy can be used to produce iPSC-CMs in suspension culture. Interestingly, it was found that high concentrations of CHIR (18 μM) are optimal for successful differentiation in iPSCs of later passages, whereas low CHIR concentrations (6 μM) are optimal for iPSCs of earlier passages (FIG. 5).

### Example 7: Upscaling

### Example 7.1: IPSC Expansion

The Inventors further aimed at showing that the developed iPSC expansion strategy can be used for upscaling into larger culture volumes. First, the UniVessel 0.5L system controlled by a Biostat B-DCU II unit was used for upscaling. Subsequently, the strategy was transferred to the UniVessel 2L system. Experiments UniVessel Familiarization Run 01 and UniVessel Proof of Concept Runs 01-08 were performed to transfer the expansion strategy established in the ambr15 system into the UniVessel 0.5L system. The proof of concept for the UniVessel 2L was shown in UniVessel Proof of Concept Run 08. UniVessel Cultivation Optimization Runs 01-09 were performed to further optimize the iPSC expansion in the Univessel 0.5L system and to adapt the passaging strategy that was established in the ambr15 to the UniVessel 0.5L system.

Importantly, culture conditions were found that support the aggregation and proliferation of iPSCs (Table 1) in the UniVessel 0.5L and 2L. These conditions were optimized in the 0.5L system and still need to be optimized for the 2L system. Contrary to the ambr15 system, the impeller blade angle is adjustable in the UniVessel system. It was found in the 0.5L system that the interplay of stirring speed and impeller blade angle has an effect on the quality of iPSCs in suspension and should be optimized further for the 2L system.

TABLE 6
Optimal culture conditions in UniVessels

| Parameter | UniVessel 0.5 L | | UniVessel 2 L | |
|---|---|---|---|---|
| Temperature | 37° | C. | 37° | C. |
| pH | 7.4 | | 7.4 | |
| Oxygen concentration | 23.8% air saturation | | 23.8% air saturation | |
| Stirring speed | d0-d1: 120 rpm | | 70 | rpm |
| | d1-end of passage: | | | |
| | 100 rpm | | | |
| Stirring direction | Downwards | | Downwards | |
| Blade angle of | 45° | | 45° | |
| impeller | | | | |
| Cultivation volume | 300-500 | mL | 500 | mL |
| Seeding density | 2.5 × 10⁵ | cells/mL | 2.5 × 10⁵ | cells/mL |
| Medium exchange | 62-100% per day with | | 50% per day with | |
| volume | spinfilter | | spinfilter | |
| Beginning of medium | Day | 2 | Day | 2 |

exchange

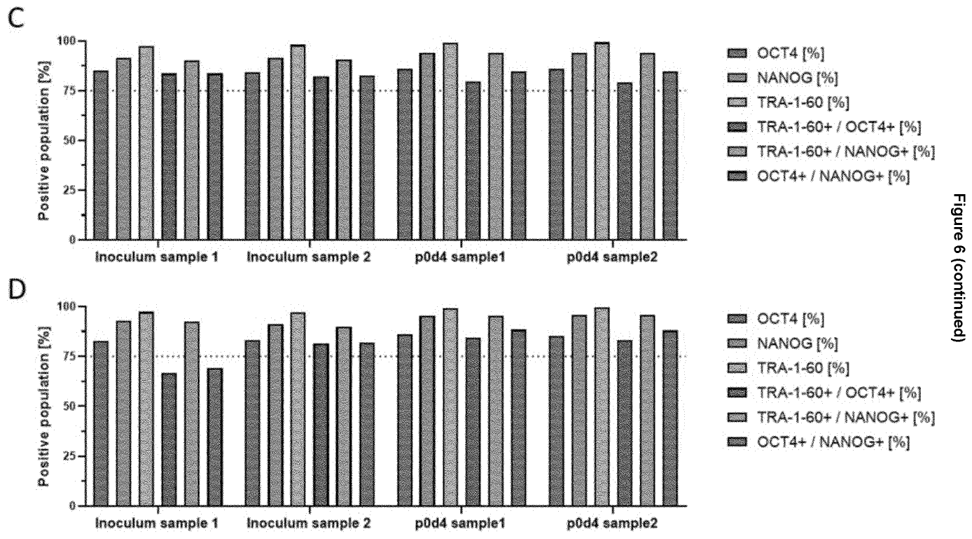

During UniVessel Proof of Concept Run 08, the UniVessel 0.5L and 2L systems were used for iPSCs suspension culture (FIG. 6). Good expansion rates of 8.78-fold increase (UniVessel 0.5 L) and 8.28-fold increase (UniVessel 2 L) were achieved. The aggregate size was smaller at day 4 in both UniVessel systems compared to aggregates cultured in the ambr15 system. This was also found in the other UniVessel experiments. Importantly, the expression of pluripotency-related markers after four days of culture was high in both the UniVessel 0.5L and 2L system.

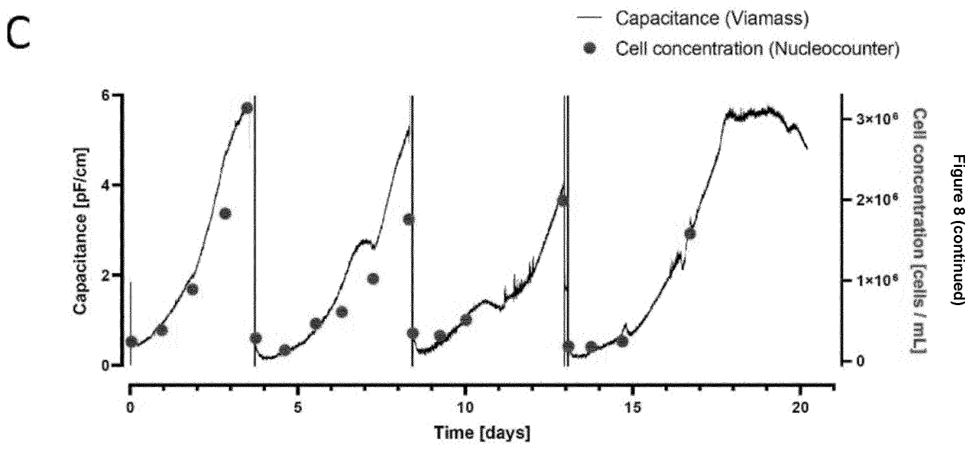

During the UniVessel Proof of Concept runs and UniVessel Cultivation Optimization runs, inline permittivity measurements were performed using the BioPat Viamass. Surprisingly, a strong correlation of capacitance and cell concentration assessed with the Nucelocounter-200 was found (FIG. 6B and FIG. 8C). The capacitance did not correlate with the aggregate size, which was expected before the experiments. The use of permittivity measurement probes in iPSCs suspension cultures was described in an invention report. Inline permittivity measurement will be a valuable tool in future applications to monitor proliferation in real-time and to control key cultivation parameters such as feeding.

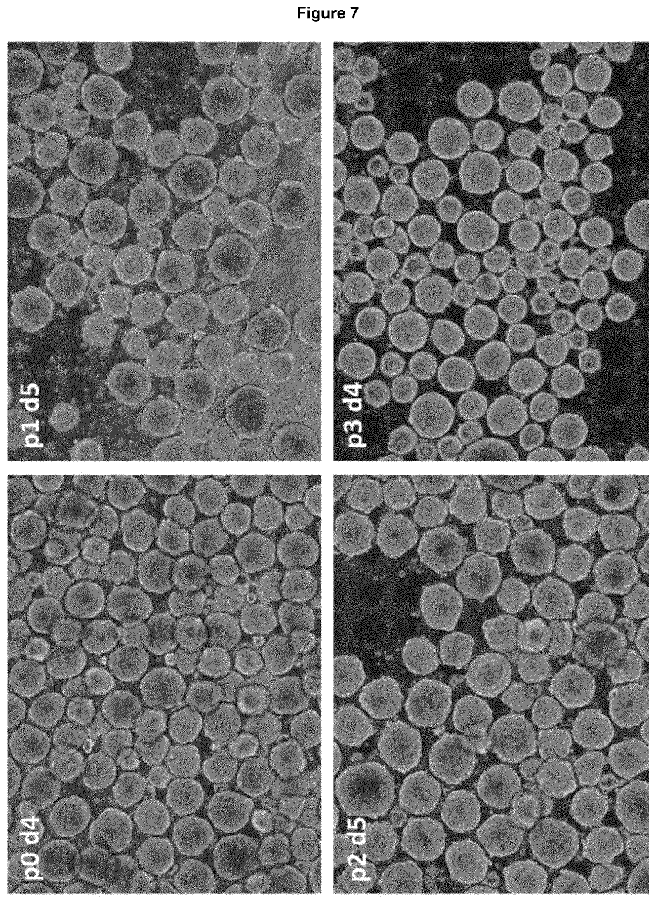

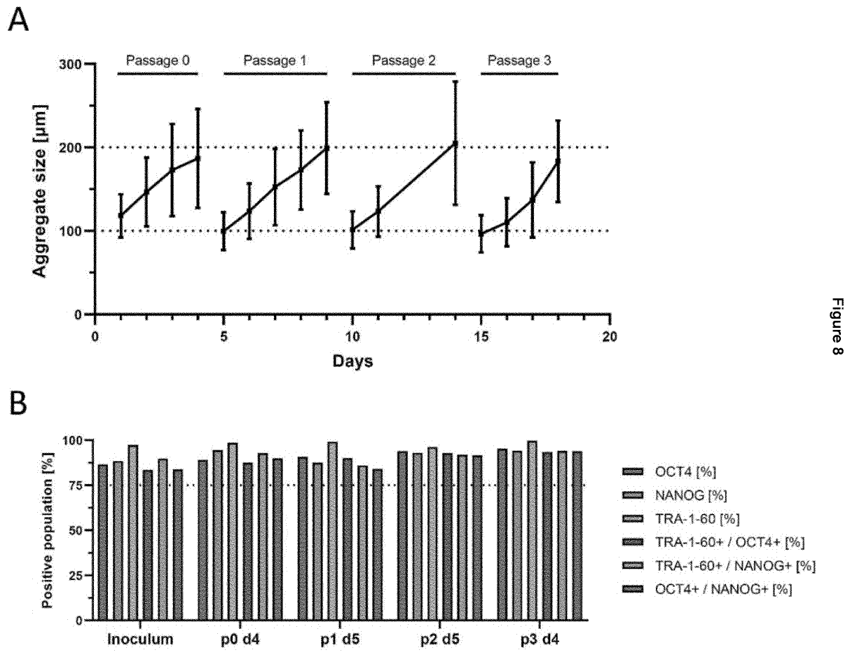

The passaging of iPSCs in the UniVessel system was also tested based on the strategy established in the ambr15. The proof of concept for the passaging and long-term culture of iPSCs in the UniVessel was shown in UniVessel Cultivation Optimization Run 09. Here, iPSCs were cultivated for 18 days in 4 passages. The aggregates had a good morphology in all passages (FIG. 7).

The expansion rates (Table 7) during long-term culture in the UniVessel 0.5L system were comparable to the long-term cultures in the ambr15 system.

TABLE 7
Expansion rates of iPSCs during long-term culture in the UniVessel 0.5 L system.

| | Day 0 | | Day 4 or 5 (end of passage) | | |
|---|---|---|---|---|---|
| | Cell concentration | Cell | Cell concentration | Cell | Expansion rate |
| Sample | [cells/mL] | number | [cells/mL] | number | [fold change] |
| Passage 0 | 2.41E+05 | 1.08E+08 | 3.14E+06 | 1.41E+09 | 13.03x |
| Passage 1 | 2.84E+05 | 9.09E+07 | 1.76E+06 | 5.63E+08 | 6.20x |
| Passage 2 | 3.45E+05 | 1.10E+08 | 1.99E+06 | 6.37E+08 | 5.77x |
| Passage 3 | 1.85E+05 | 5.92E+07 | 1.58E+06 | 5.06E+08 | 8.54x |

Furthermore, the aggregate sizes ranged from about 100 μm at day 1 of a passage to about 200 μm at the end of a passage (FIG. 8A). The same aggregate sizes were achieved with the ambr15 system. This indicates that the culture conditions established in the UniVessel can match those of the ambr15 culture. Importantly, the expression of pluripotency-related genes was very high at the end of each passage in the UniVessel (FIG. 8B). Interestingly, a trend for an increased expression can be seen with increasing passage.

During the UniVessel Cultivation Optimization Run 09, inline permittivity measurements were performed for the entirety of the run using the BioPat Viamass. The strong correlation of capacitance and cell concentration that was found in passage 0 of UniVessel Proof of Concept runs was validated in passage 1-3 (FIG. 8C). These findings highlight the potential of inline permittivity measurements for monitoring the cell concentration in real-time.

### Example 7.2. Cardiac Differentiation

One of the applications for iPSC aggregates generated in suspension culture is the largescale production of cardiomyocytes. For this purpose, the ABCF differentiation protocol should be used that was established in the Department of Pharmacology of the UMG and is used for the GMP-production of iPSC-CMs in adherent culture. Compared to the 2-factor protocol, which was used for proof of cardiac differentiation potential in the ambr15 system, the ABCF protocol relies on the growth factors Activin-A, BMP-4 and bFGF in addition to CHIR for the mesodermal induction. The cardiac differentiation in the UniVessel system was tested in UniVessel Cultivation Optimization Run 02, 09, 10 and UniVessel Cardiac Differentiation Test Run 01+02.

During early experiments it became apparent that the application of the ABCF-protocol to iPSC aggregates in the UniVessel causes massive cell death during the first days of differentiation. This cell loss results in an insufficient yield at the end of differentiation. Although an increased rate of cell death is also observed during the first days of differentiation in adherent cell culture, the yield is still high at the end of differentiation. Therefore, the observations in the suspension culture were unexpected and surprising.

It was hypothesized that the differentiation medium (BSFM) is suitable for differentiated cells but not for high-quality iPSCs in cell-only aggregates. Furthermore, the cells may adapt to the BSFM medium during differentiation. Accordingly, high-quality iPSCs in cell-only aggregates may not reach the differentiation stage necessary to survive in BSFM before the unfavorable conditions cause cell death. The hypothesis was tested in UniVessel Cultivation Optimization Run 09 and UniVessel Cardiac Differentiation Test Run 02 by applying varying ratios of iPSC-brew medium and BSFM as basal medium at differentiation start (day 0). This was done to make the transition between media types less stressful and to enable the cells to adapt to the BSFM. The differentiations were performed in agitated T25 flasks. Surprisingly, this strategy greatly increased the cell survival in all conditions tested that included iPS-brew medium on the first day of differentiation (as little amounts as 25%). This large effect was not anticipated and was much unexpected. This soft medium transition strategy should be further optimized in future experiments because it holds promise to significantly increase iPSC-CM yield.

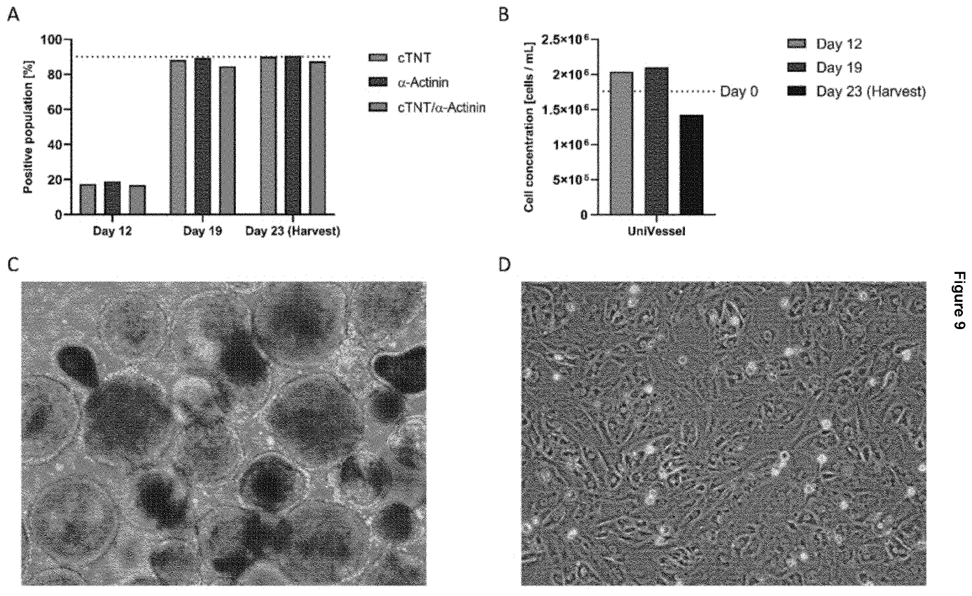

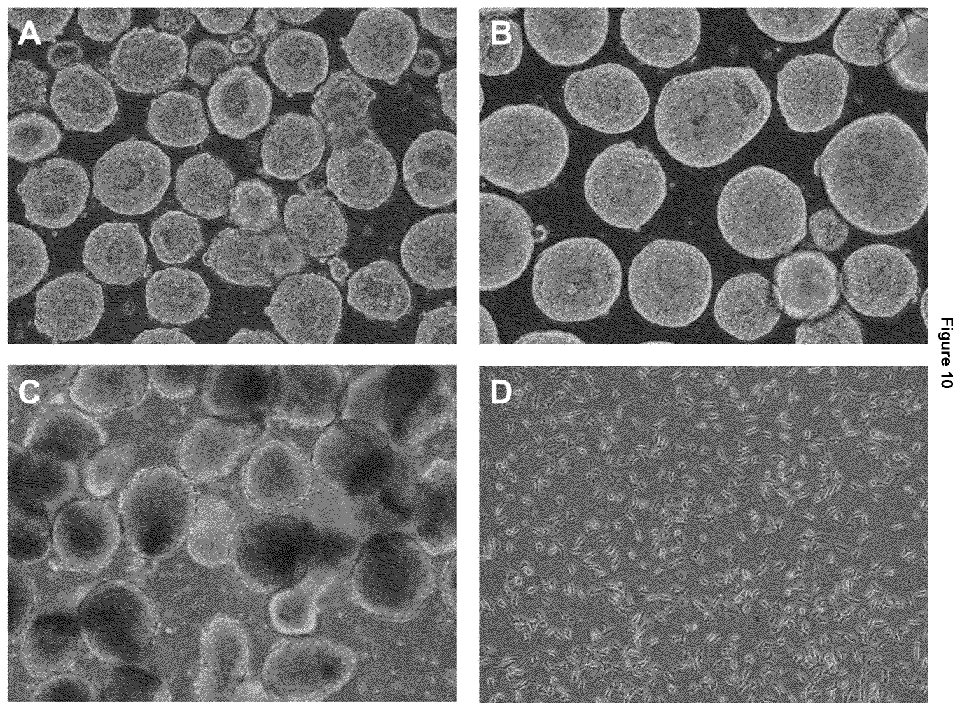

During UniVessel Cardiac Differentiation Test Run 02 the proof of concept for the cardiac differentiation of cell-only iPSC aggregates in the UniVessel 0.5L system was shown. The ABCF-protocol was used in combination with the soft medium transition strategy and an optimized strategy for washing steps and feeding. On the day of harvest (day 23) 90% of the cells expressed the cardiac markers cTNT and a-actinin (FIG. 9) and a yield of 4.26×108 living cells was achieved in a cultivation volume of 300 mL (FIG. 9B). The differentiated aggregates had a dense and compact morphology with only few cyst-like structures (FIG. 10C). After singularization and seeding onto laminin-coated plates, the iPSC-CMs attached well and depicted typical morphology (FIG. 9C). It should be highlighted that the entire culture from inoculation of iPSCs to harvest of iPSC-CMs was performed in a closed bioreactor system without the need for centrifugation or other interferences. Therefore, the developed strategy should be highly adaptable for automatization and GMP-production.

The singularization of iPSC-CMs on the day of harvest was sufficient with a viability of 88.9%. However, the collection and processing of larger volumes of cell suspension needs to be optimized because at several steps of the harvest aggregates were lost. Since no cell death was apparent between day 19 and 23, it is reasonable to assume that the decrease of cell concentration on day 23 compared to 19 was largely caused by cell loss during harvest and processing.

The iPSC-CMs generated in UniVessel Cardiac Differentiation Test 02 were used to test cryo-conservation. Different commercial and self-made freezing media were tested. The best results were achieved with CryoStor CS10 (66% viability) and Bambanker (61% viability) after 7 days of cryostorage in the vapor phase of liquid nitrogen. Importantly, the iPSC-CMs attached well to laminin-coated plates after thawing. After 7 days of adherent culture, about the same cell number that was plated could be retrieved. Both freezing media are serum-free and are available animal component-free.

### Example 8: Upscaling to 2L UniVessel

### Experimental Design and Experiment Progression

iPSCs were cultured in a 2L UniVessel for eight days (two passages) and subsequently subjected to cardiac differentiation in the same vessel for 21 days. The generated iPSC-CM aggregates were dissociated in the same vessel and singularized iPSC-CMs were harvested. Unless stated otherwise in the following, the experiments were carried out as described above. Differences include: start of cardiac differentiation on passage 1 day 4 and use of UniVessel 2L and Biostat B.

### Culture Conditions

Cells: TC1133 TL004
Targeted seeding conditions:
   Passage 0: 700 ml with 2.5×10⁵ cells/ml.
   Passage 1: 2L with 2.5×10⁵ cells/ml.
Medium change:
   iPSCs: Start at d2 of passage, perfusion 60% targeted.
   Cardiac differentiation:
      Change between medium types: 2x wash with new medium type using vessel settling (1:5 dilution in each wash). Subsequent addition of new medium type to fill up to working volume.
      Medium change d3-10; 12-19; 20-23: perfusion medium exchange of 50%/day.
Culture parameters: 37° C., pH set point 7.4, d0 set point 23.8%, 45° blade angle, 80 rpm downstirr (passage 0) and 90 rpm downstirr (passage 1 and cardiac differentiation).
   Dissociation of iPSC-CM Aggregates

At the day of harvest the iPSC-CM aggregates were dissociated in the same vessel that was used for the culture without opening the bioreactor or interfering manually. Following steps were performed for dissociation and harvest:
1. Stop agitation
2. Aggregate sedimentation (5 min)
3. Aspiration of medium to 200 ml with dip tube of fixed height
4. Addition of 10× TrypLE to 1L
5. Agitate for 10 sec. to ensure even distribution
6. Stop agitation
7. Aggregate sedimentation (5 min)
8. Aspiration of medium to 200 ml with dip tube of fixed height
9. Addition of 10× TrypLE to 1L
10. Agitate for 10 sec. to ensure even distribution
11. Stop agitation
12. Aggregate sedimentation (5 min)
13. Aspiration of medium to 200 ml with dip tube of fixed height
14. Addition of 10× TrypLE to 500 ml
15. Agitate for 50 min with 300 rpm
16. Transfer of the complete cell suspension via a dip tube, which reaches the bottom of the vessel, into 2L stop-medium (RPMI+20% Knockout-Serum Replacement)

### Materials

### Reagents and Materials:

StemMACS iPS-Brew XF, Basal Medium, Order no.: 130-107-086
StemMACS iPS-Brew XF 50× Supplement; Order no.: 130-107-087.
ROCKi: Y27632 dihydrochloride; Tocris Cat #1254
Recombinant Human/Mouse/Rat Activin A Protein; Bio-Techne; 338-AC.
Recombinant Human BMP-4; R&D Systems, 314-BP
GSK-3 Inhibitor XVI (CHIR99021); Merck; 361559-5 MG
Animal-Free Recombinant Human FGF-basic (154 a.a.); Peprotech; AF-100-18B
Stemolecule Wnt Inhibitor IWP-4; Stemgent; 04-0036
RPMI 1640 Medium, GlutaMAX Supplement; ThermoFisher Scientific; 61/870,036
TrypLE Select Enzyme (10X); ThermoFisher Scientific; A1217702
KnockOut Serum Replacement; ThermoFisher Scientific; 10828010

### Devices

Biostat B Single CC-UniVessel Glass 2L 230V
pH-Meter: Multi 3510 IDS; Xylem Analytics Germany GmbH
pH-Elektrode: SenTix Micro 900P; WTW
Nucleocounter NC-200 Type 900-0201
Flow Cytometer: CytoFlex; Beckman Coulter

### Results

During this run an 8-day iPSC culture was performed in the UniVessel, followed by a 21-day cardiac differentiation. The iPSC-CM aggregates were dissociated in the UniVessel using 10× TrypLE. Harvested iPSC-CM were plated onto Laminin.

### Aggregate/Cell Morphology:

The iPSC aggregates showed typical morphology and aggregate size was homologous within the culture (FIG. 10A and B). During differentiation the morphology of aggregates became more diffuse and heterogeneous (FIG. 10C). Contracting aggregates were observed from day 7 onward. After harvest the singularized iPSC-CM attached well to laminin-coated plates (FIG. 10D).

### Cell Quality

After eight days of expansion 5×10⁹ iPSCs of high quality were generated (Table 8).

TABLE 8
iPSC Expansion.

| Viability | 95.2% | |
|---|---|---|
| Cell concentration in 2 L | 2.5 × 10⁶ | cells/mL |
| Cell number | 5 × 10⁹ | cells |

| | |
|---|---|
| NANOG expression | 90% |
| OCT4 expression | 93% |
| TRA-1-60 expression | 97% |

After 21 days of cardiac differentiation 4.24×10⁹ iPSC-CM of high purity were harvested (Table 9). Singularization of iPSC-CM was very efficient but also gentle as indicated by the high viability of harvested cells. The singularized iPSC-CMs attached well to laminin-coated plates.

TABLE 9
iPSC-CM harvest.

| Viability | 97.2% | |
|---|---|---|
| Cell concentration in 2 L | 2.12 × 10⁶ | cells/mL |
| Cell number | 4.24 × 10⁹ | cells |

| | |
|---|---|
| α-actinin expression | 95% |

### Analysis

5×10⁹ iPSCs were generated in a 2L UniVessel and subjected to cardiac differentiation. After 21 days of differentiation 4.24×10⁹ iPSC-CM of high purity were harvested.

For the first time, the dissociation of iPSC-CM aggregates was performed in a 2L UniVessel. The viability after harvest was high and the cells attached well onto culture plates. These results show that the method of singularization is highly suitable for the harvest of iPSC-CM produced in bioreactors. These data are in line with previous dissociation tests in the amrb15. Here, it was shown that iPSC-CM aggregates can be dissociated by the combination of treatment with 10× TrypLE and mechanical force (stirring) while maintaining a high viability.

It is important to highlight that the entire run corresponding to the method of the invention was performed in a closed bioreactor from inoculation to harvest of singularized iPSC-CM. The process performed in this run is therefore highly suited for the production of large amounts of iPSC-CM for clinical applications regulated by GMP. The iPSC-CMs can be produced with a high degree of control, monitoring and automatization without manual interference or opening the closed system.

### Example 9: Summary and Discussion

In this project the small-scale suspension culture of iPSCs in the ambr15 system was successfully established and an expansion strategy for long-term cultivation was developed. Using this strategy, high quality iPSCs were produced until passage 10, which remained their cardiac differentiation potential.

Importantly, the cultivation of iPSCs in suspension can be performed automatically by the ambr15 system using the developed strategy. With regards to the automatic passaging, it was especially unexpected to find that dissociated iPSCs can reaggregate when the dissociation reagent (Versene) is diluted with fresh medium. Usually, the dissociation reagents need to be inactivated or separated from the cells to enable reattachment and proliferation.

Furthermore, it was unexpected to find that a delayed beginning of medium exchange (day 2 instead of day 1) and a reduction of exchanged medium per day (62% instead of 100%) resulted in increased iPSC yields with a remaining high quality.

Proof of concept was shown for the upscaling of the developed iPSCs suspension culture strategy in the UniVessel 0.5L and 2L systems. Optimization of cultivation in the UniVessel 0.5L resulted in high quality of iPSCs that is comparable to the results in the ambr15 system.

Finally, proof of concept for the large-scale cardiac differentiation in the UniVessel system was shown. Here, 4.26×10⁸ cells with 90% cardiomyocyte purity were generated in 300 mL culture volume.

### REFERENCES

Ast, T., and Mootha, V. K. (2019). Oxygen and mammalian cell culture: are we repeating the experiment of Dr. Ox? Nat. Metab. 1, 858-860.
Baghbaderani, B. A., Tian, X., Neo, B. H., Burkall, A., Dimezzo, T., Sierra, G., Zeng, X., Warren, K., Kovarcik, D. P., Fellner, T., et al. (2015). cGMP-Manufactured Human Induced Pluripotent Stem Cells Are Available for Pre-clinical and Clinical Applications. Stem Cell Rep. 5, 647-659.
Baghbaderani, B. A., Syama, A., Sivapatham, R., Pei, Y., Mukherjee, O., Fellner, T., Zeng, X., and Rao, M. S. (2016). Detailed Characterization of Human Induced Pluripotent Stem Cells Manufactured for Therapeutic Applications. Stem Cell Rev. Rep. 12, 394-420.
Baptista, R. P., Fluri, D. A., and Zandstra, P. W. (2013). High density continuous production of murine pluripotent cells in an acoustic perfused bioreactor at different oxygen concentrations. Biotechnol. Bioeng. 110, 648-655.
Chatterjee, P., Cheung, Y., and Liew, C. (2011). Transfecting and Nucleofecting Human Induced Pluripotent Stem Cells. J. Vis. Exp. JoVE.
Chen, V. C., Couture, S. M., Ye, J., Lin, Z., Hua, G., Huang, H.-I. P., Wu, J., Hsu, D., Carpenter, M. K., and Couture, L. A. (2012). Scalable GMP compliant suspension culture system for human ES cells. Stem Cell Research 8, 388-402.
Chen, V. C., Ye, J., Shukla, P., Hua, G., Chen, D., Lin, Z., Liu, J., Chai, J., Gold, J., Wu, J., et al. (2015). Development of a scalable suspension culture for cardiac differentiation from human pluripotent stem cells. Stem Cell Research 15, 365-375.
Correia, C., Serra, M., Espinha, N., Sousa, M., Brito, C., Burkert, K., Zheng, Y., Hescheler, J., Carrondo, M. J. T., Šarić, T., et al. (2014). Combining Hypoxia and Bioreactor Hydrodynamics Boosts Induced Pluripotent Stem Cell Differentiation Towards Cardiomyocytes. Stem Cell Rev. Rep. 10, 786-801.
Fernandes-Platzgummer, A., Diogo, M. M., Lobato da Silva, C., and Cabral, J. M. S. (2014). Maximizing mouse embryonic stem cell production in a stirred tank reactor by controlling dissolved oxygen concentration and continuous perfusion operation. Biochem. Eng. J. 82, 81-90.
Forsyth, N. R., Musio, A., Vezzoni, P., Simpson, A. H. R. W., Noble, B. S., and McWhir, J. (2006). Physiologic oxygen enhances human embryonic stem cell clonal recovery and reduces chromosomal abnormalities. Cloning Stem Cells 8, 16-23.
Halloin, C., Coffee, M., Manstein, F., and Zweigerdt, R. (2019). Production of Cardiomyocytes from Human Pluripotent Stem Cells by Bioreactor Technologies. In Cell-Based Assays Using IPSCs for Drug Development and Testing, C.-F. Mandenius, and J. A. Ross, eds. (New York, NY: Springer New York), pp. 55-70.
Hemmi, N., Tohyama, S., Nakajima, K., Kanazawa, H., Suzuki, T., Hattori, F., Seki, T., Kishino, Y., Hirano, A., Okada, M., et al. (2014). A Massive Suspension Culture System With Metabolic Purification for Human Pluripotent Stem Cell-Derived Cardiomyocytes. STEM CELLS Translational Medicine 3, 1473-1483.
Jiang, Y., Langenberg, K., Borgdorff, V., Duńska, M., Post, R., Bartulos, O., Doornbos, M., Braam, S., Reijerkerk, A., and Rasche, U. (2019). Controlled, Large-Scale Manufacturing of hiPSC-Derived Cardiomyocytes in Stirred-Tank Bioreactors. 12.
Kempf, H., Kropp, C., Olmer, R., Martin, U., and Zweigerdt, R. (2015). Cardiac differentiation of human pluripotent stem cells in scalable suspension culture. Nature Protocols 10, 1345-1361.
Kropp, C., Kempf, H., Halloin, C., Robles-Diaz, D., Franke, A., Scheper, T., Kinast, K., Knorpp, T., Joos, T. O., Haverich, A., et al. (2016). Impact of Feeding Strategies on the Scalable Expansion of Human Pluripotent Stem Cells in Single-Use Stirred Tank Bioreactors. STEM CELLS Translational Medicine 5, 1289-1301.
Kropp, C., Massai, D., and Zweigerdt, R. (2017). Progress and challenges in large-scale expansion of human pluripotent stem cells. Process Biochemistry 59, 244-254.
Le, M. N. T., and Hasegawa, K. (2019). Expansion Culture of Human Pluripotent Stem Cells and Production of Cardiomyocytes. Bioengineering 6, 48.
Loh, K. M., Chen, A., Koh, P. W., Deng, T. Z., Sinha, R., Tsai, J. M., Barkal, A. A., Shen, K. Y., Jain, R., Morganti, R. M., et al. (2016). Mapping the pairwise choices leading from pluripotency to human bone, heart and other mesoderm cell-types. Cell 166, 451-467.
Niebruegge, S., Bauwens, C. L., Peerani, R., Thavandiran, N., Masse, S., Sevaptisidis, E., Nanthakumar, K., Woodhouse, K., Husain, M., Kumacheva, E., et al. (2009). Generation of human embryonic stem cell-derived mesoderm and cardiac cells using size-specified aggregates in an oxygen-controlled bioreactor. Biotechnol. Bioeng. 102, 493-507.
Nogueira, D. E. S., Rodrigues, C. A. V., Carvalho, M. S., Miranda, C. C., Hashimura, Y., Jung, S., Lee, B., and Cabral, J. M. S. (2019). Strategies for the expansion of human induced pluripotent stem cells as aggregates in single-use Vertical-Wheel™ bioreactors. J. Biol. Eng. 13, 74.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat. Methods 9, 676-682.
Serra, M., Brito, C., Sousa, M. F. Q., Jensen, J., Tostões, R., Clemente, J., Strehl, R., Hyllner, J., Carrondo, M. J. T., and Alves, P. M. (2010). Improving expansion of pluripotent human embryonic stem cells in perfused bioreactors through oxygen control. J. Biotechnol. 148, 208-215.
Tiburcy, M., Hudson, J. E., Balfanz, P., Schlick, S., Meyer, T., Chang Liao, M.-L., Levent, E., Raad, F., Zeidler, S., Wingender, E., et al. (2017). Defined Engineered Human Myocardium With Advanced Maturation for Applications in Heart Failure Modeling and Repair. Circulation 135, 1832-1847.
Voisard, D., Meuwly, F., Ruffieux, P.-A., Baer, G., and Kadouri, A. (2003). Potential of cell retention techniques for large-scale high-density perfusion culture of suspended mammalian cells. Biotechnol. Bioeng. 82, 751-765.
Zhang, J., Tao, R., Campbell, K. F., Carvalho, J. L., Ruiz, E. C., Kim, G. C., Schmuck, E. G., Raval, A. N., Rocha, A. M. da, Herron, T. J., et al. (2019). Functional cardiac fibroblasts derived from human pluripotent stem cells via second heart field progenitors. Nat. Commun. 10, 1-15.

1. A method of producing a population of differentiated cells from pluripotent stem cells (PSC) in suspension culture in a bioreactor, the method comprising
(i) cultivating the PSC under suitable conditions to allow mesodermal induction,
(ii) inducing differentiation under suitable conditions of the PSC of step (i), and
(iii) optionally selecting the PSC of step (ii),
wherein steps (i) to (iii) are conducted in a closed bioreactor system, thereby producing a population of differentiated cells.

2. The method of claim 1, wherein the method comprises a further step (0) of expanding PSC under suitable conditions.

3. The method of claim 1 or 2, wherein after step (ii) a further step (ii) (a) is conducted of expanding the PSC of step (ii) under suitable conditions.

4. The method of any one of the preceding claims, wherein after step (iii) a further step (iv) is conducted of recovery of the PSC of step (iii) under suitable conditions in the closed bioreactor system.

5. The method of any one of the preceding claims, wherein after step (ii), (iii) or (iv) a further step (v) is conducted of harvesting of the population of differentiated cells.

6. The method of claim 5, wherein harvesting of the population of differentiated cells includes dissociating aggregates formed during any one of steps (i) to (iv).

7. The method of claim 6, wherein dissociating aggregates includes:
(a) allowing the cells or aggregates to sediment at the bottom of the closed bioreactor system and removing the supernatant;
(b) adding a cell dissociation agent, preferably an enzyme such as trypsin;
(c) agitating the cells or cell aggregates;
(d) repeating steps (a)-(c) three times; and
(e) stopping the cell dissociation by adding stop medium, preferably wherein the stop medium comprises Knockout-Serum replacement.

8. The method of any of the preceding claims, wherein the differentiated cells are cardiomyocytes.

9. The method of any one of the preceding claims, wherein enriching in step (iii) comprises conducting metabolic selection under suitable conditions.

10. The method of any one of the preceding claims, wherein the closed bioreactor system is a stirred bioreactor, a rocking motion bioreactor and/or a multi parallel bioreactor.

11. The method of any one of the preceding claims, wherein
(a) the medium is
in step (0) iPS-brew basal medium comprising iPS-brew supplement;
in step (i) a mesodermal induction medium (MIM), which comprises RPMI-1640, about 1 to 5% B27 minus insulin, about 100 to 300 μmol/L I-ascorbic acid-2-phosphate sesquimagnesium salt hydrate, about 0.1 to 10 mM sodium pyruvate, about 5 to 15 ng/ml Activin A, about 1 to 10 ng/ml BMP4, about 1 to 10 ng/ml bFGF, optionally about 1 to 3 μM CHIR99021;
in step (ii) a cardiomyocyte induction medium (CM-IM) comprising RPMI-1640, about 1 to 5% B27, about 100 to 300 μmol/L I-ascorbic acid-2-phosphate sesquimagnesium salt hydrate, about 0.1 to 10 mM sodium pyruvate, about 1 to 15 μM IWP4;
in step (iii) a selection medium comprising RPMI-1640 minus glucose, about 1 to 5 mM Lactate, about 0.01 to 0.5 mM 2-Mercaptoethanol, about 2 to 8 mM HEPES; and
in step (iv) a basal serum-free medium (BSFM), which comprises RPMI-1640, about 1 to 5% B27, about 100 to 300 μmol/L L-ascorbic acid-2-phosphate sesquimagnesium salt hydrate, and about 0.1 to 10 mM sodium pyruvate; and/or
(b) the steps are conducted for the following periods
step (0) optionally about 4 to 6 days,
step (i) about 24 hours to 5 days,
step (ii) about 4 to 10 days,
step (ii) (a) about 1 to 5 days,
step (iii) about 5 to 10 days,
step (iv) about 12 hours to 5 days.

12. The method of any one of the preceding claims, wherein a change of the medium is conducted at least at the transition from step (0) to step (i), from step (i) to step (ii), from step (ii) to step (ii) (a), from step (ii) (a) to step (iii), and from step (iii) to step (iv), wherein the change of the medium preferably includes a step of washing the cells comprised in the closed bioreactor system.

13. The method of any one of the preceding claims, wherein the medium change is a complete or a partial medium change;
wherein the change of medium from step (0) to step (i) comprises a partial medium change, preferably wherein the medium of step (0) remains unchanged for an amount of about 5 to 75% v/v, preferably 25% v/v; and/or
wherein step (0), step (ii), step (ii) (a), step (iii) and step (iv) are conducted comprising perfusion of the medium.

14. The method of any one of the preceding claims, wherein cell aggregates formed during step (0) are dissociated within the closed bioreactor system.

15. The method of any one of the preceding claims, wherein the differentiated cells are selected from the group consisting of cardiomyocytes, skeletal muscle cells, fibroblasts, stromal cells, endothelial cells, and leukocytes.

16. A population of differentiated cells obtainable or obtained by the method of any one of claims 1 to 15.
